(19) United States
(12) Patent Application Publication
(10) Pub. No.: US 2022/0378764 A1
(43) Pub. Date: Dec. 1, 2022

(54) MODULATING EXPRESSION LEVEL OF A GENE ENCODING A CYTOCHROME P450 PROTEIN BY TREATING A HUMAN SUBJECT WITH A NITROXIDE

(71) Applicant: Louis HABASH, Irvine, CA (US)

(72) Inventor: Louis HABASH, Irvine, CA (US)

(21) Appl. No.: 17/330,122

(22) Filed: May 25, 2021

(51) Int. Cl. A61K 31/445; A61K 45/06

(52) U.S. Cl. CPC A61K 31/445; A61K 45/06

(57) ABSTRACT

A method of treatment is disclosed. The method comprises administering to a human subject having or at risk of developing a disease associated with decreased expression of one or more genes encoding cytochrome P450, an effective amount of a nitroxide antioxidant, wherein the nitroxide antioxidant increases an expression level of one or more genes encoding cytochrome p450 enzymes.

### BACKGROUND

### Field

The present disclosure relates generally to the field of therapeutic modification of gene expression and more particularly to treating human subjects with diseases identified by respective gene expression, with a nitroxide antioxidant.

### Description of the Related Art

Diseases and conditions are treatable by adjusting the expression levels and activities of key genes in the body. Gene expression irregularities, whether overexpressed, activated, under expressed or inhibited underlie the development and progression of diseases and conditions. Some diseases are characterized by deficient expression of certain genes while other diseases result from over expression of certain genes. A disease resulting from irregular gene expression can be prevented, treated, or reversed by administering a nitroxide antioxidant to target and correct the expression levels of the genes.

Expression levels of genes are often naturally controlled in an appropriate way, but sometimes natural control of gene expression fails. For example, in cancer, genes providing instructions for cell growth are activated or switched on, when they should be off. Autoimmune diseases and aging are other examples of diseases and conditions that result from irregular gene expression. As cells age, the natural control of gene expression deteriorates promoting several diseases and conditions such as inflammation, chronic pain, infections, neurodegenerative disease, neurological disorders, skin diseases, etc. It is essential to identify the irregular expression of the genes involved in the cause of the disease and adjust the expression levels of those genes.

Often referred to as gene therapy, the targeting and correction of cellular dysfunction through adjusting the expression level of certain genes is necessary to prevent, treat, or reverse a disease or condition. Only by identifying key genes and developing therapeutics that altering the expression patterns of those genes can we prevent the development of the disease, reduce its effects once it has occurred, or reverse it all together.

One of the key gene families involved in several diseases and conditions is cytochrome p450 (CYP450). When this gene is underexpressed it causes several diseases and conditions associated with the underexpression of the gene. Thus, correction of the underexpression of CYP450 genes is essential for treatment and prevention of the associated diseases and conditions.

### SUMMARY

Some embodiments disclosed herein provide methods for increasing gene expression. The methods, in some embodiments, include identifying a human subject over the age of 35 and having a decrease expression level of a gene associated with the cytochrome p450 family; and administering to the human subject an effective amount of a nitroxide antioxidant resulting in an increased expression level of the gene. In some embodiments, the gene is selected from the group consisting of: Cyp2c29, Cyp3a25, Cyp3a11, Cyp2j5, Cyp2c50, Cyp2c55, Cyp2d9, Cyp2e1, Cyp2b9, Cyp3a13, Cyp4f15, Cyp2c70, Cyp46a1, Cyp27a1, Cyp4v3, and homologues thereof. In some embodiments, the human subject is over the age of 45. In some embodiments, the human subject is over the age of 55. In some embodiments, the human subject is over the age of 65. In some embodiments, the expression level of the gene in a skin tissue is increased. In some embodiments, the expression level of the gene in an adipose tissue is increased. In some embodiments, the expression level of the gene in blood is increased. In some embodiments, the expression level of the gene in a neuronal tissue is increased. In some embodiments, the expression level of the gene in cardiac tissue is increased. In some embodiments, the nitroxide antioxidant is 4-hydroxy-2,2,6,6-tetramethylpiperidine-1-oxyl. In some embodiments, the effective amount of the nitroxide antioxidant is within a range of 0.01-300 mg/kg. In some embodiments, the effective amount of the nitroxide antioxidant is within a range of 0.1-250 mg/kg. In some embodiments, the effective amount of the nitroxide antioxidant is within a range of 1-200 mg/kg. In some embodiments, the effective amount of the nitroxide antioxidant is within a range of 2-150 mg/kg. In some embodiments, the effective amount of the nitroxide antioxidant is within a range of 5-125 mg/kg. In some embodiments, the effective amount of the nitroxide antioxidant is within a range of 7-100 mg/kg. In some embodiments, the effective amount of the nitroxide antioxidant is within a range of 10-75 mg/kg. In some embodiments, the effective amount of the nitroxide antioxidant is within a range of 15-30 mg/kg.

Some embodiments disclosed herein provide methods for increasing the expression level of a gene in a human subject in need thereof, comprising: identifying a human subject having a decreased expression level of a gene associated with the cytochrome p450 family; administering to the human subject an effective amount of a nitroxide antioxidant, whereby the expression level of the gene associated with the cytochrome p450 family is increased. In some embodiments, the gene is selected from the group consisting of: Cyp2c29, Cyp3a25, Cyp3a11, Cyp2j5, Cyp2c50, Cyp2c55, Cyp2d9, Cyp2e1, Cyp2b9, Cyp3a13, Cyp4f15, Cyp2c70, Cyp46a1, Cyp27a1, Cyp4v3, and homologues thereof. In some embodiments, the decreased expression level of the gene is age-related. In some embodiments, the human subject is over the age of 35. In some embodiments, the human subject is over the age of 45. In some embodiments, the human subject is over the age of 55. In some embodiments, the human subject is over the age of 65. In some embodiments, the decreased expression level of the gene is disease-related. In some embodiments, the disease is selected from the group consisting of cancer, rheumatoid/osteoid arthritis, systemic lupus erythematosus (SLE), inflammatory bowel disease, Alzheimer's disease, multiple sclerosis, atherosclerosis, cardiovascular disease, cataracts, dementia, osteoporosis, type 2 diabetes, and hypertension. In some embodiments, the disease is age-related. In some embodiments, the expression level of the gene in a skin tissue is increased. In some embodiments, the expression level of the gene in an adipose tissue is increased. In some embodiments, the expression level of the gene in blood is increased. In some embodiments, the expression level of the gene in a neuronal tissue is increased. In some embodiments, the expression level of the gene in cardiac tissue is increased. In some embodiments, the nitroxide antioxidant is 4-hydroxy-2,2,6,6-tetramethylpiperidine-1-oxyl. In some embodiments, the effective amount of the nitroxide antioxidant is within a range of 0.01-300 mg/kg. In some embodiments, the effective amount of the nitroxide antioxidant is within a range of 0.1-250 mg/kg. In some embodiments, the effective amount of the nitroxide antioxidant is within a range of 1-200 mg/kg. In some embodiments, the effective amount of the nitroxide antioxidant is within a range of 2-150 mg/kg. In some embodiments, the effective amount of the nitroxide antioxidant is within a range of 5-125 mg/kg. In some embodiments, the effective amount of the nitroxide antioxidant is within a range of 7-100 mg/kg. In some embodiments, the effective amount of the nitroxide antioxidant is within a range of 10-75 mg/kg. In some embodiments, the effective amount of the nitroxide antioxidant is within a range of 15-30 mg/kg.

Some embodiments disclosed herein provide methods for reducing risk of a disease in a human subject in need thereof, comprising: identifying a human subject over the age of 35 having an increased risk of a disease due to a decreased expression level of a gene associated with the cytochrome p450 family; administering to the human subject an effective amount of a nitroxide antioxidant, whereby the expression level of the gene associated with the cytochrome p450 family is increased. In some embodiments, the disease is selected from the group consisting of cancer, rheumatoid/osteoid arthritis, systemic lupus erythematosus (SLE), inflammatory bowel disease, Alzheimer's disease, multiple sclerosis, atherosclerosis, cardiovascular disease, cataracts, dementia, osteoporosis, type 2 diabetes, and hypertension. In some embodiments, the gene is selected from the group consisting of: Cyp2c29, Cyp3a25, Cyp3a11, Cyp2j5, Cyp2c50, Cyp2c55, Cyp2d9, Cyp2e1, Cyp2b9, Cyp3a13, Cyp4f15, Cyp2c70, Cyp46a1, Cyp27a1, Cyp4v3, and homologues thereof. In some embodiments, the human subject is over the age of 45. In some embodiments, the human subject is over the age of 55. In some embodiments, the human subject is over the age of 65. In some embodiments, the expression level of the gene in a skin tissue is increased. In some embodiments, the expression level of the gene in an adipose tissue is increased. In some embodiments, the expression level of the gene in blood is increased. In some embodiments, the expression level of the gene in a neuronal tissue is increased. In some embodiments, the expression level of the gene in cardiac tissue is increased. In some embodiments, the nitroxide antioxidant is 4-hydroxy-2,2,6,6-tetramethylpiperidine-1-oxyl. In some embodiments, the effective amount of the nitroxide antioxidant is within a range of 0.01-300 mg/kg. In some embodiments, the effective amount of the nitroxide antioxidant is within a range of 0.1-250 mg/kg. In some embodiments, the effective amount of the nitroxide antioxidant is within a range of 1-200 mg/kg. In some embodiments, the effective amount of the nitroxide antioxidant is within a range of 2-150 mg/kg. In some embodiments, the effective amount of the nitroxide antioxidant is within a range of 5-125 mg/kg. In some embodiments, the effective amount of the nitroxide antioxidant is within a range of 7-100 mg/kg. In some embodiments, the effective amount of the nitroxide antioxidant is within a range of 10-75 mg/kg. In some embodiments, the effective amount of the nitroxide antioxidant is within a range of 15-30 mg/kg.

Some embodiments disclosed herein provide methods comprising: identifying a human subject having or at risk of developing a cancer and in need of an increased expression level of a gene associated with the cytochrome p450 family; administering to the human subject an effective amount of a nitroxide antioxidant, whereby the expression level of the gene associated with the cytochrome p450 family is increased. In some embodiments, the cancer can be selected from the group consisting of renal cell carcinoma bladder cancer, colorectal cancer, hepatocellular carcinoma, prostate carcinoma, and kidney carcinoma. In some embodiments, the gene is selected from the group consisting of: Cyp2c29, Cyp3a25, Cyp3a11, Cyp2j5, Cyp2c50, Cyp2c55, Cyp2d9, Cyp2e1, Cyp2b9, Cyp3a13, Cyp4f15, Cyp2c70, Cyp46a1, Cyp27a1, Cyp4v3, and homologues thereof. In some embodiments, the cancer is age-related. In some embodiments, the human subject is over the age of 35. In some embodiments, the human subject is over the age of 45. In some embodiments, the human subject is over the age of 55. In some embodiments, the human subject is over the age of 65. In some embodiments, the expression level of the gene in a skin tissue is increased. In some embodiments, the expression level of the gene in an adipose tissue is increased. In some embodiments, the expression level of the gene in blood is increased. In some embodiments, the expression level of the gene in a neuronal tissue is increased. In some embodiments, the expression level of the gene in cardiac tissue is increased. In some embodiments, the nitroxide antioxidant is 4-hydroxy-2,2,6,6-tetramethylpiperidine-1-oxyl. In some embodiments, the effective amount of the nitroxide antioxidant is within a range of 0.01-300 mg/kg. In some embodiments, the effective amount of the nitroxide antioxidant is within a range of 0.1-250 mg/kg. In some embodiments, the effective amount of the nitroxide antioxidant is within a range of 1-200 mg/kg. In some embodiments, the effective amount of the nitroxide antioxidant is within a range of 2-150 mg/kg. In some embodiments, the effective amount of the nitroxide antioxidant is within a range of 5-125 mg/kg. In some embodiments, the effective amount of the nitroxide antioxidant is within a range of 7-100 mg/kg. In some embodiments, the effective amount of the nitroxide antioxidant is within a range of 10-75 mg/kg. In some embodiments, the effective amount of the nitroxide antioxidant is within a range of 15-30 mg/kg.

Some embodiments disclosed herein provide methods comprising: identifying a human subject having or at risk of developing an autoimmune disease and in need of an increased expression level of a gene associated with the cytochrome p450 family; administering to the human subject an effective amount of a nitroxide antioxidant, wherein the expression level of the gene associated with the cytochrome p450 family is increased. In some embodiments, the autoimmune disease can be selected from the group consisting of rheumatoid/osteoid arthritis, systemic lupus erythematosus (SLE), inflammatory bowel disease, multiple sclerosis, atherosclerosis, and osteoporosis. In some embodiments, the gene is selected from the group consisting of: Cyp2c29, Cyp3a25, Cyp3a11, Cyp2j5, Cyp2c50, Cyp2c55, Cyp2d9, Cyp2e1, Cyp2b9, Cyp3a13, Cyp4f15, Cyp2c70, Cyp46a1, Cyp27a1, Cyp4v3, and homologues thereof. In some embodiments, the gene is Gstm3. In some embodiments, the autoimmune disease is age-related. In some embodiments, the human subject is over the age of 35. In some embodiments, the human subject is over the age of 45. In some embodiments, the human subject is over the age of 55. In some embodiments, the human subject is over the age of 65. In some embodiments, the expression level of the gene in a skin tissue is increased. In some embodiments, the expression level of the gene in an adipose tissue is increased. In some embodiments, the expression level of the gene in blood is increased. In some embodiments, the expression level of the gene in a neuronal tissue is increased. In some embodiments, the expression level of the gene in a cardiac tissue is increased. In some embodiments, the nitroxide antioxidant is 4-hydroxy-2,2,6,6-tetramethylpiperidine-1-oxyl. In some embodiments, the effective amount of the nitroxide antioxidant is within a range of 0.01-300 mg/kg. In some embodiments, the effective amount of the nitroxide antioxidant is within a range of 0.1-250 mg/kg. In some embodiments, the effective amount of the nitroxide antioxidant is within a range of 1-200 mg/kg. In some embodiments, the effective amount of the nitroxide antioxidant is within a range of 2-150 mg/kg. In some embodiments, the effective amount of the nitroxide antioxidant is within a range of 5-125 mg/kg. In some embodiments, the effective amount of the nitroxide antioxidant is within a range of 7-100 mg/kg. In some embodiments, the effective amount of the nitroxide antioxidant is within a range of 10-75 mg/kg. In some embodiments, the effective amount of the nitroxide antioxidant is within a range of 15-30 mg/kg.

Some embodiments disclosed herein provide methods for a disease associated with a decreased apoptosis in a patient in need thereof, comprising: identifying a human subject having or at risk of developing a disease associated with a decreased expression of a gene associated with the cytochrome p450 family; administering to the human subject an effective amount of a nitroxide antioxidant, whereby the expression level of a gene associated with the cytochrome p450 family is increased. In some embodiments, the disease can be selected from the group consisting of cancer, rheumatoid/osteoid arthritis, systemic lupus erythematosus (SLE), inflammatory bowel disease, Alzheimer's disease, multiple sclerosis, atherosclerosis, cardiovascular disease, cataracts, dementia, osteoporosis, type 2 diabetes, and hypertension. In some embodiments, the gene is selected from the group consisting of: Cyp2c29, Cyp3a25, Cyp3a11, Cyp2j5, Cyp2c50, Cyp2c55, Cyp2d9, Cyp2e1, Cyp2b9, Cyp3a13, Cyp4f15, Cyp2c70, Cyp46a1, Cyp27a1, Cyp4v3, and homologues thereof. In some embodiments, the human subject is over the age of 35. In some embodiments, the human subject is over the age of 45. In some embodiments, the human subject is over the age of 55. In some embodiments, the human subject is over the age of 65. In some embodiments, the expression level of the gene in a skin tissue is increased. In some embodiments, the expression level of the gene in an adipose tissue is increased. In some embodiments, the expression level of the gene in blood is increased. In some embodiments, the expression level of the gene in a neuronal tissue is increased. In some embodiments, the expression level of the gene in a cardiac tissue is increased. In some embodiments, the nitroxide antioxidant is 4-hydroxy-2,2,6,6-tetramethylpiperidine-1-oxyl. In some embodiments, the effective amount of the nitroxide antioxidant is within a range of 0.01-300 mg/kg. In some embodiments, the effective amount of the nitroxide antioxidant is within a range of 0.1-250 mg/kg. In some embodiments, the effective amount of the nitroxide antioxidant is within a range of 1-200 mg/kg. In some embodiments, the effective amount of the nitroxide antioxidant is within a range of 2-150 mg/kg. In some embodiments, the effective amount of the nitroxide antioxidant is within a range of 5-125 mg/kg. In some embodiments, the effective amount of the nitroxide antioxidant is within a range of 7-100 mg/kg. In some embodiments, the effective amount of the nitroxide antioxidant is within a range of 10-75 mg/kg. In some embodiments, the effective amount of the nitroxide antioxidant is within a range of 15-30 mg/kg.

Some embodiments disclosed herein provide methods for treating an individual in need thereof, comprising: identifying an individual over the age of 35 in need of an increased expression level of a gene associated with the cytochrome p450 family; and administering to the individual an effective amount of a nitroxide antioxidant to increase the level of expression of the gene associated with the cytochrome p450 family. In some embodiments, the gene is selected from the group consisting of: Cyp2c29, Cyp3a25, Cyp3a11, Cyp2j5, Cyp2c50, Cyp2c55, Cyp2d9, Cyp2e1, Cyp2b9, Cyp3a13, Cyp4f15, Cyp2c70, Cyp46a1, Cyp27a1, Cyp4v3, and homologues thereof. In some embodiments, the human subject is over the age of 45. In some embodiments, the human subject is over the age of 55. In some embodiments, the human subject is over the age of 65. In some embodiments, the human subject has a decrease expression level of the gene. In some embodiments, the individual has or is at risk of developing an age-related condition. In some embodiments, the age-related condition comprises increased senescence in a tissue. In some embodiments, the age-related condition comprises inactivation of one or more cytochrome p450 enzymes in a tissue. In some embodiments, the age-related condition comprises increased molecular heterogeneity. In some embodiments, the age-related condition comprises increased functional impairment in a tissue. In some embodiments, the expression level of the gene in a skin tissue is increased. In some embodiments, the expression level of the gene in an adipose tissue is increased. In some embodiments, the expression level of the gene in blood is increased. In some embodiments, the expression level of the gene in a neuronal tissue is increased. In some embodiments, the nitroxide antioxidant is 4-hydroxy-2,2,6,6-tetramethylpiperidine-1-oxyl. In some embodiments, the effective amount of the nitroxide antioxidant is within a range of 0.01-300 mg/kg. In some embodiments, the effective amount of the nitroxide antioxidant is within a range of 0.1-250 mg/kg. In some embodiments, the effective amount of the nitroxide antioxidant is within a range of 1-200 mg/kg. In some embodiments, the effective amount of the nitroxide antioxidant is within a range of 2-150 mg/kg. In some embodiments, the effective amount of the nitroxide antioxidant is within a range of 5-125 mg/kg. In some embodiments, the effective amount of the nitroxide antioxidant is within a range of 7-100 mg/kg. In some embodiments, the effective amount of the nitroxide antioxidant is within a range of 10-75 mg/kg. In some embodiments, the effective amount of the nitroxide antioxidant is within a range of 15-30 mg/kg.

Some embodiments disclosed herein provide methods for treating an individual in need thereof, comprising: identifying an individual having a disease-related decreased expression level of a gene associated with the cytochrome p450 family; and administering to the individual an effective amount of a nitroxide antioxidant to increase the level of expression of the gene associated with the cytochrome p450 family. In some embodiments, the disease can be selected from the group consisting of cancer, rheumatoid/osteoid arthritis, systemic lupus erythematosus (SLE), inflammatory bowel disease, Alzheimer's disease, multiple sclerosis, atherosclerosis, cardiovascular disease, cataracts, dementia, osteoporosis, type 2 diabetes, and hypertension. In some embodiments, the gene is selected from the group consisting of: Cyp2c29, Cyp3a25, Cyp3a11, Cyp2j5, Cyp2c50, Cyp2c55, Cyp2d9, Cyp2e1, Cyp2b9, Cyp3a13, Cyp4f15, Cyp2c70, Cyp46a1, Cyp27a1, Cyp4v3, and homologues thereof. In some embodiments, the human subject is over the age of 35. In some embodiments, the human subject is over the age of 45. In some embodiments, the human subject is over the age of 55. In some embodiments, the human subject is over the age of 65. In some embodiments, the expression level of the gene in a skin tissue is increased. In some embodiments, the expression level of the gene in an adipose tissue is increased. In some embodiments, the expression level of the gene in blood is increased. In some embodiments, the expression level of the gene in a neuronal tissue is increased. In some embodiments, the expression level of the gene in a cardiac tissue is increased. In some embodiments, the nitroxide antioxidant is 4-hydroxy-2,2,6,6-tetramethylpiperidine-1-oxyl. In some embodiments, the effective amount of the nitroxide antioxidant is within a range of 0.01-300 mg/kg. In some embodiments, the effective amount of the nitroxide antioxidant is within a range of 0.1-250 mg/kg. In some embodiments, the effective amount of the nitroxide antioxidant is within a range of 1-200 mg/kg. In some embodiments, the effective amount of the nitroxide antioxidant is within a range of 2-150 mg/kg. In some embodiments, the effective amount of the nitroxide antioxidant is within a range of 5-125 mg/kg. In some embodiments, the effective amount of the nitroxide antioxidant is within a range of 7-100 mg/kg. In some embodiments, the effective amount of the nitroxide antioxidant is within a range of 10-75 mg/kg. In some embodiments, the effective amount of the nitroxide antioxidant is within a range of 15-30 mg/kg.

Some embodiments disclosed herein provide methods for treating an individual having or at risk of developing a condition due to aging, comprising: identifying an individual over the age of 35; and administering to the individual an effective amount of a nitroxide antioxidant, whereby the expression level of the gene associated with the cytochrome p450 family is increased. In some embodiments, the individual has a decreased expression level of the gene. In some embodiments, the gene is selected from the group consisting of: Cyp2c29, Cyp3a25, Cyp3a11, Cyp2j5, Cyp2c50, Cyp2c55, Cyp2d9, Cyp2e1, Cyp2b9, Cyp3a13, Cyp4f15, Cyp2c70, Cyp46a1, Cyp27a1, Cyp4v3, and homologues thereof. In some embodiments, the condition is an age-related condition. In some embodiments, the age-related condition comprises increased senescence in a tissue. In some embodiments, the age-related condition comprises inactivation of one or more cytochrome p450 enzymes in a tissue. In some embodiments, the age-related condition comprises increased molecular heterogeneity. In some embodiments, the age-related condition comprises increased functional impairment in a tissue. In some embodiments, the age- related condition is selected from the group consisting of cancer, rheumatoid/osteoid arthritis, systemic lupus erythematosus (SLE), inflammatory bowel disease, Alzheimer's disease, multiple sclerosis, atherosclerosis, cardiovascular disease, cataracts, dementia, osteoporosis, type 2 diabetes, and hypertension. In some embodiments, the human subject is over the age of 35. In some embodiments, the human subject is over the age of 45. In some embodiments, the human subject is over the age of 55. In some embodiments, the human subject is over the age of 65.

Some embodiments disclosed herein provide methods for treatment, comprising administering to a human subject, known to have decreased glutathione activity, an effective amount of a nitroxide antioxidant, wherein the nitroxide antioxidant increases an expression level of one or more genes encoding cytochrome p450 enzymes, thereby increasing glutathione activity. In some embodiments, the human subject is further known to have a disease in which the expression level of at least one gene selected from the group consisting of Cyp2c29, Cyp3a25, Cyp3a11, Cyp2j5, Cyp2c50, Cyp2c55, Cyp2d9, Cyp2e1, Cyp2b9, Cyp3a13, Cyp4f15, Cyp2c70, Cyp46a1, Cyp27a1, Cyp4v3, and homologues thereof. In some embodiments, the nitroxide antioxidant is 4-hydroxy-2,2,6,6-tetramethylpiperidine-1-oxyl. In some embodiments, the nitroxide antioxidant increases GST enzymatic activity. In some embodiments, the disease is defined by elevated cellular toxicity mediated by one or more xenobiotics. In some embodiments, the one or more genes are selected from a group consisting of Cyp2c29, Cyp3a25, Cyp3a11, Cyp2j5, Cyp2c50, Cyp2c55, Cyp2d9, Cyp2e1, Cyp2b9, Cyp3a13, Cyp4f15, Cyp2c70, Cyp46a1, Cyp27a1, Cyp4v3, and homologues thereof.

Some embodiments disclosed herein provide methods for inhibiting development of a cancer, comprising administering to a human subject, known to be at risk of developing cancer mediated by decreased xenobiotic metabolism, an effective amount of a nitroxide antioxidant, wherein xenobiotic metabolism is increased, thereby inhibiting development of said cancer. In some embodiments, the human subject exhibits no outward symptoms of said cancer. In some embodiments, the human subject is not known to have said cancer. In some embodiments, the human subject is further known to have a decreased expression level of one or more genes selected from the group consisting of Cyp2c29, Cyp3a25, Cyp3a11, Cyp2j5, Cyp2c50, Cyp2c55, Cyp2d9, Cyp2e1, Cyp2b9, Cyp3a13, Cyp4f15, Cyp2c70, Cyp46a1, Cyp27a1, Cyp4v3, and homologues thereof, and the treatment increases said expression level. In some embodiments, the nitroxide antioxidant is 4-hydroxy-2,2,6,6-tetramethylpiperidine-1-oxyl. In some embodiments, the nitroxide antioxidant increases enzyme mediated Xenobiotic metabolism.

Some embodiments disclosed herein provide methods for increasing the expression level of a gene in a human subject in need thereof, comprising identifying a human subject having a decreased expression level of a gene associated with GST activity, wherein the gene is selected from the group consisting of Cyp2c29, Cyp3a25, Cyp3a11, Cyp2j5, Cyp2c50, Cyp2c55, Cyp2d9, Cyp2e1, Cyp2b9, Cyp3a13, Cyp4f15, Cyp2c70, Cyp46a1, Cyp27a1, Cyp4v3, and homologues thereof; and administering to the human subject an effective amount of a nitroxide antioxidant to increase the level of expression of the gene associated with GST. In some embodiments, the nitroxide antioxidant is 4-hydroxy-2,2,6,6-tetramethylpiperidine-1-oxyl. In some embodiments, the decreased expression level of the gene is age-related. In some embodiments, the decreased expression level of the gene is associated with cancer. In some embodiments, the decreased expression level of the gene is associated with a disease. In some embodiments, the decreased expression level of the gene is associated with a neurodegenerative disorder. In some embodiments, the decreased expression level of the gene is associated with an infection. In some embodiments, the decreased expression level of the gene is associated with an oxidative stress. In some embodiments, the expression level of the gene is reduced in a tissue selected from the group consisting of a skin tissue, an immune tissue, an adipose tissue, a pancreatic tissue, cardiac tissue, and a neuronal tissue.

Some embodiments disclosed herein provide methods for increasing an expression level, in a eukaryotic cell, of one or more genes encoding one or more GST enzymes by administering a nitroxide antioxidant to eukaryotic cell. In some embodiments, the one or more genes is selected from the group consisting of Cyp2c29, Cyp3a25, Cyp3a11, Cyp2j5, Cyp2c50, Cyp2c55, Cyp2d9, Cyp2e1, Cyp2b9, Cyp3a13, Cyp4f15, Cyp2c70, Cyp46a1, Cyp27a1, Cyp4v3, and homologues thereof. In some embodiments, the nitroxide antioxidant is 4-hydroxy-2,2,6,6-tetramethylpiperidine-1-oxyl. In some embodiments, the eukaryotic cell is a cancer cell. In some embodiments, the expression level of the one or more genes is decreased in a tissue selected from the group consisting of a skin tissue, an immune tissue, an adipose tissue, a pancreatic tissue, cardiac tissue, and a neuronal tissue. In some embodiments, the expression level of the one or more genes is age-related. In some embodiments, the expression level of the one or more genes is disease-related. In some embodiments, the expression level of the one or more genes is neurodegenerative-related.

Some embodiments disclosed herein provide methods for treating a condition comprising identifying an individual known to have a condition mediated by xenobiotic toxicity administering to the individual an effective amount of a nitroxide antioxidant to whereby an expression level of a one or more cytochrome p450 enzymes is increased. In some embodiments, the gene is selected from the group consisting of Cyp2c29, Cyp3a25, Cyp3a11, Cyp2j5, Cyp2c50, Cyp2c55, Cyp2d9, Cyp2e1, Cyp2b9, Cyp3a13, Cyp4f15, Cyp2c70, Cyp46a1, Cyp27a1, Cyp4v3, and homologues thereof. In some embodiments, the nitroxide antioxidant is 4-hydroxy-2,2,6,6-tetramethylpiperidine-1-oxyl. In some embodiments, the condition is age-related. In some embodiments, the condition is cancer. In some embodiments, the condition is a disease. In some embodiments, the condition is a neurodegenerative disorder. In some embodiments, the condition is an infection. In some embodiments, the condition is associated with an oxidative stress. In some embodiments, the condition is chemotherapy-induced toxicity. In some embodiments, the condition is radiation-induced toxicity. In some embodiments, the xenobiotic toxicity is caused by oxidative stress.

Some embodiments disclosed herein provide methods for ameliorating xenobiotic induced oxidative stress comprising administering an effective amount of a nitroxide antioxidant to an individual known to have xenobiotic induced oxidative stress whereby an expression level of one or more GST genes is upregulated. In some embodiments, the gene is selected from the group consisting of Cyp2c29, Cyp3a25, Cyp3a11, Cyp2j5, Cyp2c50, Cyp2c55, Cyp2d9, Cyp2e1, Cyp2b9, Cyp3a13, Cyp4f15, Cyp2c70, Cyp46a1, Cyp27a1, Cyp4v3, and homologues thereof. In some embodiments, the nitroxide antioxidant is 4-hydroxy-2,2,6,6-tetramethylpiperidine-1-oxyl. In some embodiments, the xenobiotic induced oxidative stress is age-related. In some embodiments, the xenobiotic induced oxidative stress is related to cancer. In some embodiments, the xenobiotic induced oxidative stress is related a disease. In some embodiments, the xenobiotic induced oxidative stress is related to neurodegenerative disorder. In some embodiments, the xenobiotic induced oxidative stress is related to infection. In some embodiments, the xenobiotic induced oxidative stress is related to one or more exogenous factors. In some embodiments, the xenobiotic induced oxidative stress is related to one or more endogenous factors. In some embodiments, the individual has been administered a chemotherapeutic agent. In some embodiments, the nitroxide antioxidant is chemically attached to one or more bioeffector molecules.

Some embodiments disclosed herein provide methods for preventing a condition comprising identifying an individual at risk of xenobiotic toxicity administering to the individual an effective amount of a nitroxide antioxidant to whereby an expression level of one or more cytochrome p450 enzymes is increased .In some embodiments, the gene is selected from the group consisting of Cyp2c29, Cyp3a25, Cyp3a11, Cyp2j5, Cyp2c50, Cyp2c55, Cyp2d9, Cyp2e1, Cyp2b9, Cyp3a13, Cyp4f15, Cyp2c70, Cyp46a1, Cyp27a1, Cyp4v3, and homologues thereof. In some embodiments, the nitroxide antioxidant is 4-hydroxy-2,2,6,6-tetramethylpiperidine-1-oxyl. In some embodiments, the condition is age-related. In some embodiments, the condition is cancer. In some embodiments, the condition is a disease. In some embodiments, the condition is a neurodegenerative disorder. In some embodiments, the condition is an infection. In some embodiments, the condition is associated with an oxidative stress. In some embodiments, the xenobiotic toxicity is chemotherapy-induced. In some embodiments, the xenobiotic toxicity is radiation-induced. In some embodiments, the xenobiotic toxicity is caused by oxidative stress. In some embodiments, the individual has been administered or exposed to a xenobiotic, wherein the effective amount of a nitroxide antioxidant is administered prior to the administration or exposure to the xenobiotic. In some embodiments, the individual has been administered or exposed to a xenobiotic, wherein the effective amount of a nitroxide antioxidant is administered in conjunction with the administration or exposure to the xenobiotic.

### DETAILED DESCRIPTION

### Definitions

Unless defined otherwise, technical and scientific terms used herein have the same meaning as commonly understood by one of ordinary skill in the art to which the present disclosure belongs. See, e.g. Singleton et al., Dictionary of Microbiology and Molecular Biology 2nd ed., J. Wiley & Sons (New York, N.Y. 1994); Sambrook et al., Molecular Cloning, A Laboratory Manual, Cold Springs Harbor Press (Cold Springs Harbor, N.Y. 1989). For purposes of the present disclosure, the following terms are defined below.

All patents, applications, published applications and other publications referred to herein are incorporated by reference for the referenced material and in their entireties. If a term or phrase is used herein in a way that is contrary to or otherwise inconsistent with a definition set forth in the patents, applications, published applications and other publications that are herein incorporated by reference, the use herein prevails over the definition that is incorporated herein by reference.

As used herein, the term “expression” means the detection of a gene product that is expressed or produced by a nucleic acid molecule by standard molecular biology methods, which gene product refers to e.g. an unspliced RNA, an mRNA, a splice variant mRNA, a polypeptide, a post-translationally modified polypeptide, a splice variant polypeptide etc., and specifically products made using an RNA gene product as a template, e.g. cDNA of the RNA.

As used herein, “differential expression” of a gene means that the expression of the gene is at a higher level (“decreased expression”) or lower level (“decreased expression”) in a human subject suffering from a disease, for example cancers and autoimmune diseases, relative to its expression in a normal or control subject. Differential expression includes both quantitative, as well as qualitative, differences in the temporal or cellular expression pattern in a gene or its expression products among, for example, normal and diseased cells, or among cells which have undergone different disease events or disease stages.

As used herein, “increasing the expression level” of a gene means causing the expression of the gene to decrease by treating the human subject with a compound, for example a nitroxide antioxidant, such that the expression level of the gene after treatment is lower than the expression level of the gene before treatment in the human subject.

As used herein, “delivering” a compound shall mean bringing that compound into contact with a relevant cell, tissue, or organism. Similarly, “contacting” shall mean that the compound contacts a relevant target, such as a tissue or cell or tumor. In either case, delivery or contact in an organism is affected by directly administering the compound to the organism, or by administering a different compound to the organism, such as a prodrug that is converted in vivo to the desired compound. In short, these terms cover any action that leads to contact between the desired compound and a target cell, tissue, or organism.

The present disclosure describes methods of modulating gene expression in human subjects. However, this is illustrative only and not intended to be limiting. For example, the methods disclosed herein can be used for modulating gene expression in other vertebrates, such as but not limited to mammals, birds, reptiles, fish, and the like (with modifications wherein appropriate). Mammals and birds include most agricultural animals. Treatment of companion animals, e.g., dogs, cats, or birds is also contemplated.

It is understood that aspects and embodiments of the invention described herein include “consisting” and/or “consisting essentially of” aspects and embodiments.

Other objects, advantages and features of the present invention will become apparent from the following specification taken in conjunction with the accompanying drawings.

### Human Subject Identification

The present disclosure relates to methods of treating alteration in gene expression, such as age-related, cancer-related, disease-related, neurodegeneration-related, and infection-related alteration in gene expression. Gene expression changes also play important roles in aging and serve as biomarkers of physiological decline and disease conditions, such as neurodegenerative diseases, and cancers. Therefore, one aspect of the present disclosure is methods of treating a human subject having an age-related, cancer-related, disease-related, neurodegeneration related, and/or infection-related decrease in gene expression levels, such as those genes associated with uncoupling proteins and uncoupling protein activity. In some embodiments, the human subject can be identified based on the human subject's age, gene expression level, family history, health conditions, medical history, habits, or a combination thereof.

Regardless of the cause of the upregulation, some common terminology can be used. In some embodiments, the expression level of a gene (e.g., a gene associated with cytochrome p450) in a human subject is considered to be downregulated or decreased if the decrease in the expression level of that gene is statistically significant compared to that of a control or a reference. In some embodiments, the expression level of a gene (e.g., a gene associated with cytochrome p450) in a human subject is considered to be downregulated or decreased if the decrease in the expression level of that gene is statistically significant compared to that of a control or a reference.

In some embodiments, a normal healthy population or a population at large can be a population having the same or similar gender, age, and/or race, compared to the human subject. In some embodiments, the expression level of the gene in the control or reference can be the mean or median expression level of the gene in control subjects in the control or reference subjects in the reference. The decrease in expression level can be statistically significant if the probability of the observed difference occurring not by chance, the confidence level, is greater than a threshold. The threshold can be, or be about, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.9%, or a number or a range between any two of these values.

In some embodiments, the decrease in expression level can be, or be about, 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 20%, 30%, 40%, 50%, 60%, 70%, 80%, 90%, 100%, or a number or a range between any two of these values. In some embodiments, the decrease in expression level can be at least 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 20%, 30%, 40%, 50%, 60%, 70%, 80%, 90%, 100%, or more.

In some embodiments, the human subject may have an age that is, is about, or is over 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 years old.

In some embodiments, the human subject is identified based on the human subject's expression profiles of a gene associated with cytochrome p450. Non-limiting exemplary methods for determining the human subject's expression profiles include: amplification techniques such as PCR and RT-PCR (including quantitative variants), hybridization techniques such as in situ hybridization, microarrays, blots, and others, and high throughput sequencing techniques like Next Generation Sequencing (Illumina, Roche Sequencer, Life Technologies SOLID™), Single Molecule Real Time Sequencing (Pacific Biosciences), True Single Molecule Sequencing (Helicos), or sequencing methods using no light emitting technologies but other physical methods to detect the sequencing reaction or the sequencing product, like Ion Torrent (Life Technologies). Non-limiting exemplary methods for determining the human subject's expression profiles include: binding techniques such as ELISA, immunohistochemistry, microarray and functional techniques such as enzymatic assays.

### Targeted Gene Expression Adjustment

Cytochrome P450 (CYP450) are a family of enzymes responsible for metabolism and biosynthesis of compounds. Regulation of their concentration and activity is essential for the treatment and prevention of diseases and conditions. Additionally, recent developments in personalized medicine consider disease treatment and management based on unique dysfunction in gene expression for each individual rather than generic treatment based on a diagnosis.

Diseases with complex pathologies such as Alzheimer's, cancer, and hepatic diseases are most appropriately treated by tailoring therapeutics with having known impact on the underlying dysfunctional gene expression. Understanding the biological process leading to a diagnosis of a disease is the necessary and the most effective way to provide disease modifying treatments compared to general treatments directed to abating one or more symptoms. The biological process begins with the regulation of protein formation through modulating the protein encoding gene or set of genes.

All living organisms are comprised of cells that function individually as well as in combination with other cells to form larger and more complex structures such as tissue and organs. The operation of each cell is based on the genetic instructions provided by the DNA contained therein. DNA is arranged in a particular sequence referred to as a gene which is transcribed and translated into a functional product required for the operation of the cell.

Genes are expressed in a particular quantity based on the instruction provided by the DNA. In particular, gene expression describes transcription of gene encoding DNA sequences into complementary DNA (cDNA) and translation of cDNA into the functional products, such as proteins. Many factors, both internal and external, are involved in regulation of gene expression in cells. Such regulation manifests in an adjustment of gene expression to increase or decrease a number of proteins made.

The quantity of expression for a particular gene or group of complementary genes can be considered relative to a healthy state or disease state of the cell. In a healthy state, genes are expressed in a predictable quantity necessary for the operation of the cell. In a disease state, the genes are overexpressed or under expressed relative to the healthy-state expression. The deviation from the healthy state of gene expression results in catastrophic burden on the cell due to over or under production of the functional product encoded by the gene.

Therefore, a condition or disease is identifiable based on such dysfunctional expression of genes within the cell. Whether the dysfunctional expression of the genes is due external influence on the cell or genetic aberrations, correction to the dysfunctional expression is necessary to address the underlying cause of the condition or disease. Overexpression or under expression of a gene or genes often results in dysfunction of downstream actions controlled by the same. Whether the gene is a regulator of cellular function or a vital in a responsive mechanism, modulation of the gene expression is a fundamental directive in addressing the foundational issues associated with many diseases and conditions.

Developing tailored treatment for a disease relies on understanding what genes are over- or underexpressed and their involvement in the development of the disease. Treatments for a disease or condition are often directed at addressing a manifestation or symptom of the disease. However, the underlying disease is permitted to remain resulting in subsequent presentation of the previously treated symptoms. It is essential to correct or reinforce the underlying cause of the disease. Ultimately, the treatment of the disease or condition requires targeting and modulating the expression level of the gene or genes that are inappropriately overexpressed or under expressed.

Inhibition or suppression of the CYP450 enzymes is responsible for diseases and conditions, such as neurodegenerative diseases, cardiovascular diseases, hepatic diseases, obesity, and aging. Underexpression of cytochrome p450 encoding genes is directly related to the inhibition, inactivity, and disfunction of vital cellular processes within cells and tissues. Thus, targeted increase of the gene expression provides a beneficial therapeutic response for the treatment and prevention of associated diseases and conditions.

### Genes Associated with the Cytochrome P450 Family

In some embodiments, administering to the human subject the effective amount of the nitroxide antioxidant results in an increased expression level of a gene, for example a gene associated with the cytochrome p450 family. Therefore, some embodiments disclosed herein provide methods for treating an individual in need thereof, comprising identifying an individual having a disease-related decreased expression level of a gene associated with the cytochrome p450 family; and administering to the individual an effective amount of a nitroxide antioxidant to increase the level of expression of the gene associated with the cytochrome p450 family. Some embodiments disclosed herein provide methods for treating an individual in need thereof, comprising identifying an individual in need of an increased expression level of a gene associated with the cytochrome p450 family; and administering to the individual an effective amount of a nitroxide antioxidant to increase the level of expression of the gene associated with the cytochrome p450 family.

Non-limiting examples of diseases associated with and altered level of the cytochrome p450 family include cancer; breast cancer; lung cancer; kidney cancer; cancers of the ovary and uterus; cancer of the central nervous system; cancers of the head and neck; melanoma; lymphomas; leukemia; neurological disorders; Alzheimer's disease; Parkinson's disease; Huntington's disease; amyotrophic lateral sclerosis; stroke; cardiovascular disorders; ischemia; heart failure; infectious diseases; bacterial infections; viral infections; autoimmune diseases; systemic lupus erythematosus; autoimmune lymphoproliferative syndrome; rheumatoid arthritis; and thyroiditis.

Non-limiting exemplary genes involved in intracellular xenobiotic metabolism activity include those involved in the cytochrome p450 family (Cyp2c29, Cyp3a25, Cyp3a11, Cyp2j5, Cyp2c50, Cyp2c55, Cyp2d9, Cyp2e1, Cyp2b9, Cyp3a13, Cyp4f15, Cyp2c70, Cyp46a1, Cyp27a1, Cyp4v3, and homologues thereof).

The gene associated with the cytochrome p450 family can be Cyp2c29, Cyp3a25, Cyp3a11, Cyp2j5, Cyp2c50, Cyp2c55, Cyp2d9, Cyp2e1, Cyp2b9, Cyp3a13, Cyp4f15, Cyp2c70, Cyp46a1, Cyp27a1, or Cyp4v3CYP2C9 Cyp3A4 Cyp3A5 Cyp3A7 Cyp3A5P1 Cyp3A43 Cyp2J2 Cyp2C18 Cyp2C19 Cyp2C8 Cyp2B7P1 Cyp2B6 Cyp4F3 Cyp4F2 Cyp4F11 Cyp4F12 Cyp2C18 Cyp2C19 Cyp46A1 Cyp4V2 KlkB1. For example, the treatment results in increased expression levels of Cyp2c29, Cyp3a25, Cyp3a11, Cyp2j5, Cyp2c50, Cyp2c55, Cyp2d9, Cyp2e1, Cyp2b9, Cyp3a13, Cyp4f15, Cyp2c70, Cyp46a1, Cyp27a1, or Cyp4v3CYP2C9 Cyp3A4 Cyp3A5 Cyp3A7 Cyp3A5P1 Cyp3A43 Cyp2J2 Cyp2C18 Cyp2C19 Cyp2C8 Cyp2B7P1 Cyp2B6 Cyp4F3 Cyp4F2 Cyp4F11 Cyp4F12 Cyp2C18 Cyp2C19 Cyp46A1 Cyp4V2 KlkB1, or any combination thereof. The increased expression levels of Cyp2c29, Cyp3a25, Cyp3a11, Cyp2j5, Cyp2c50, Cyp2c55, Cyp2d9, Cyp2e1, Cyp2b9, Cyp3a13, Cyp4f15, Cyp2c70, Cyp46a1, Cyp27a1, Cyp4v3 Cyp2C9 Cyp3A4 Cyp3A5 Cyp3A7 Cyp3A5P1 Cyp3A43 Cyp2J2 Cyp2C18 Cyp2C19 Cyp2C8 Cyp2B7P1 Cyp2B6 Cyp4F3 Cyp4F2 Cyp4F11 Cyp4F12 Cyp2C18 Cyp2C19 Cyp46A1 Cyp4V2 KlkB1, or any combination thereof, can increase xenobiotic metabolism. The increased level of xenobiotic metabolism results in a diminished cellular toxicity and thereby a decrease in or disappearance of signs and symptoms of a disease associated with a decreased expression of one or more genes of the cytochrome p450 family, including the curing of the disease associated with decreased expression of one or more genes of the cytochrome p450 family.

### Cytochrome p450 Gene Family

There are 18 mammalian cytochrome P450 (CYP) families, which encode 57 genes in the human genome. CYP2, CYP3 and CYP4 families contain far more genes than the other 15 families; these three families are also the ones that are dramatically larger in rodent genomes. Most genes in the CYP1, CYP2, CYP3 and CYP4 families encode enzymes involved in eicosanoid metabolism and are inducible by various environmental stimuli (i.e. diet, chemical inducers, drugs, pheromones, etc.), whereas the other 14 gene families often have only a single member, and are rarely if ever inducible or redundant. Although the CYP2 and CYP3 families can be regarded as largely redundant and promiscuous, mutations or other defects in one or more genes of the remaining 16 gene families are primarily the ones responsible for P450-specific diseases-confirming these genes are not superfluous or promiscuous but rather are more directly involved in critical life functions. P450-mediated diseases comprise those caused by: aberrant steroidogenesis; defects in fatty acid, cholesterol and bile acid pathways; vitamin D dysregulation and retinoid (as well as putative eicosanoid) dysregulation during fertilization, implantation, embryogenesis, foetogenesis and neonatal development. Human cytochromes P450 in health and disease I Request PDF. Available from: https://www.researchgate.net/publication/234087119_Human_cytochromes_P450_in_health_and_disease [accessed Jan. 22 2018].

The activity and impact of gene associated with the cytochrome p450 may involve consideration for genetic homologues for a particular member of the CYP450 gene family. For example, most human CYPP450 genes have one homologous mouse gene, while some human CYPP450 genes have multiple mouse homologues. For examples, CYP2A13 is homologous to Cyp2a4 and 2a5, CYP2C8 to Cyp2c66 and 2c65, CYP3A4 to Cyp3a11, 3a41a, 3a41b, and 3a44, CYP3A43 to Cyp3a25 and 3a57, whereas CYP4A11 is homologous to Cyp4a10 and 4a32. Only one mouse homolog was selected for each human P450 gene, with the exception of two Cyp3a, Cyp3a11, and Cyp3a41b, for CYP3A4, which encodes the most important P450 enzyme for xenobiotic metabolism. Other examples of CYP450 human homologues are CYP2c29 to CYP2c9; CYP3a25 to CYP3A4, CYP3A5, CYP3A7, and CYP3A5P1; CYP3a11 to CYP3A4, CYP3A7, CYP3A5P1, CYP3A43, and CYP3A5; CYP2j5 to CYP2J2; CYP2c50 to CYP2C8, CYP2C18, CYP2C19, and CYP2C9; CYP2c55 to CYP2C18, CYP2C19, CYP2C9, and CYP2C8; CYP2b9 to CYP2B7P1 and CYP2B6; CYP3A13 to CYP3A5, CYP3A4, CYP3A7, and CYP3A5P1; CYP4F15 to CYP4F3, CYP4F2, CYP4F11, and CYP4F12; CYP2c70 to CYP2C18, CYP2C19, CYP2C8, and CYP2C9; and CYP4v3 to CYP4V2 and KLKB1.

### Neurodegenerative Disease

Neurodegenerative diseases are generally characterized by the progressive degeneration of the structure and function of the central nervous system or peripheral nervous system. In particular, the progression is related to a degeneration and death of neuronal cells. A direct association between impaired cholesterol metabolism in the brain and neurodegeneration has been clearly demonstrated in several neurodegenerative disease states. CYP46A1, also known as 24-hydroxylase is directly involved in the regulation and removal of excess cholesterol buildup in the brain. Cholesterol molecules are too large to freely cross the blood-brain barrier (BBB), and therefore require efficient CYP46A1 activity to maintain a healthy state of brain function.

The BBB is a physical and metabolic barrier comprised of brain microvascular endothelial cells that restrict the passage of substances from the blood to the brain and help maintain brain homeostasis. The BBB expresses a high number of ion channels and transporters, has a low rate of pinocytosis, and forms intercellular tight junction protein complexes that limit paracellular permeability. (Lochhead, Jeffrey J et al. “Oxidative stress increases blood-brain barrier permeability and induces alterations in occludin during hypoxia-reoxygenation.” Journal of cerebral blood flow and metabolism : official journal of the International Society of Cerebral Blood Flow and Metabolism vol. 30,9 (2010): 1625-36. doi:10.1038/jcbfm.2010.29). Furthermore, studies have shown that nitroxide antioxidants such as Tempol readily crosses the BBB, thereby directly directly confirming a beneficial impact on the gene expression and protein activity therein.

Studies have shown that synthesis of new cholesterol and the secretion of 24(S)-hydroxycholesterol, a metabolite of CYP46A1, are closely coupled and that at least 40% of cholesterol turnover in the brain is dependent on the action of cholesterol 24-hydroxylase. (Lund E G, Xie C, Kotti T, Turley S D, Dietschy J M, Russell D W. Knockout of the cholesterol 24-hydroxylase gene in mice reveals a brain-specific mechanism of cholesterol turnover. J Biol Chem. 2003 Jun. 20; 278(25): 22980-8. doi: 10.1074/jbc.M303415200. Epub 2003 Apr. 9. PMID: 12686551).

Cholesterol has a remarkably long half-life in the brain (4-6 months in rodents and up to 5 years in humans) (Dietschy and Turley, 2001). In general, there is a low rate of cholesterol synthesis in the adult brain, and cholesterol cannot be degraded in the central nervous system (CNS). The conversion of cholesterol to 24-hydroxycholesterol, by the enzyme cholesterol 24-hydroxylase that is expressed in neurons, represents a major mechanism by which excess cholesterol is eliminated from the brain. Unlike cholesterol, 24-hydroxycholesterol can cross the blood-brain barrier, enter the peripheral circulation and be eliminated from the body in bile. (Dysregulation of cholesterol balance in the brain: contribution to neurodegenerative diseases. Jean E. Vance, Disease Models & Mechanisms 2012 5: 746-755; doi: 10.1242/dmm.010124).

### Alzheimer's Disease

Alzheimer's disease (AD) is a steadily growing global epidemic. Estimates suggest more than 47 million people worldwide were affected in 2015 and a staggering 131 million is predicted 30 years from now. AD is a neurodegenerative disorder characterized mainly by the loss of memory functions and accompanied by other symptoms in a wide range of classes from mood, verbalization to motor problems. The most striking outcome from this type of dementia is the incremental disability for performing everyday life routines and increasing dependence from others for care. Aging is the main risk factor for developing AD, and the risk of developing AD dementia becomes even higher as life expectancy increases and the world population becomes older.

AD has a notably complex pathology. A particular pathway to the development and progression of AD involves unregulated cholesterol concentrations inside the brain. For example, Tangles of Tau are observed In Niemann-Pick-C, a genetic disease of cholesterol metabolism, confirming the direct connection between dysfunction of cholesterol in the brain and the tangles of Tau (Loera-Valencia, R, Cedazo-Minguez, A, Kenigsberg, P, et. al. Current and emerging avenues for Alzheimer's disease drug targets (Review). J Intern Med 2019; 286: 398-437). Increased concentrations of cholesterol are attributed to the development and progression of AD. Cholesterol is directly associated with plaques and tangles. In vitro and in vivo studies have shown that increased cholesterol content in membranes is associated with increased A-beta production. Conversely, decreased cholesterol in the membranes favours the nonamyloidogenic pathway of APP processing. (Loera-Valencia, 2019).

The inability for regulated cholesterol turnover and excretion from the brain causes and promotes neuron cell death. The inability to remove cholesterol may be attributed to increased amyloid processing proteins or increased deposition of amyloid placques, both of which result in decreased expression and activity of CYP46A1, the enzyme responsible for cholesterol turnover and remove across the blood-brain barrier. Current treatment methods are generally limited by their ability to effectively address the underlying causes of AD on the opposite side of the blood-brain barrier.

CYP450 enzymes are known to be ubiquitously expressed, and in particular in the brain. In particular, cytochrome P450 46A1 (CYP46A1 or cholesterol 24-hydroxylase) controls cholesterol elimination from the brain and plays a role in higher order brain functions. (Natalia Mast, Aicha Saadane, Ana Valencia-Olvera, James Constans, Erin Maxfield, Hiroyuki Arakawa, Young Li, Gary Landreth, Irina A. Pikuleva, Cholesterol-metabolizing enzyme cytochrome P450 46A1 as a pharmacologic target for Alzheimer's disease, Neuropharmacology, Volume 123, 2017, Pages 465-476, ISSN 0028-3908, https://doi.org/10.1016/j.neuropharm.2017.06.026.). CYP46A1 catalyzes cholesterol 24-hydroxylation, the major mechanism for cholesterol removal from the brain. Normally, CYP46A1 resides in the endoplasmic reticulum and is expressed in neurons of the hippocampus, cortex, and cerebellum. In Alzheimer's disease, however, CYP46A1 shows prominent expression in astrocytes and around amyloid plaques. (Mast, 2017).

By increasing the expression of CYP450 enzymes such as CYP46A1, cholesterol turnover in improved and excess cholesterol is removed from the brain thereby preventing and treating the development and progression of AD. Alternatively, CYP46A1 provides an indicator of increased susceptibility to developing AD. Patients having a family history can benefit from the evaluation of CYP46A1 expression to identify diminished capacity and increased risk of AD development.

### Neimann-Pick Disease

A direct association between impaired cholesterol metabolism in the brain and neurodegeneration has been clearly demonstrated in several neurodegenerative diseases. In particular, Neimann-Pick type C (NPC) disease.

NPC disease is an autosomal recessive inherited disorder that causes progressive neurodegeneration and premature death, and is often accompanied by hepatosplenomegaly and lung disease. A characteristic histological feature of brains of individuals with NPC disease is a massive loss of neurons, particularly Purkinje cells in the cerebellum, consistent with the impairment of motor function in these individuals. While NPC disease pathology involves deficient NPC proteins, the deficiency in these proteins results in decreased transportation of cholesterol within the brain. (Vance, 2012). Increasing CYP46A1 is associated with increased CYP46A1 activity, including the increased metabolism and excretion of cholesterol and can provide a compensatory mechanism to prevent or inhibit the progression of NPC disease.

### Huntington's Disease

Huntington's disease is an autosomal dominant neurodegenerative disease caused by abnormal polyglutamine expansion in huntingtin leading to degeneration of striatal neurons. Altered brain cholesterol homeostasis is implicated in Huntington's disease, with increased accumulation of cholesterol in striatal neurons. Huntington's disease is also associated with abnormalities in cholesterol metabolism, as observed in other neurodegenerative diseases including Niemann-Pick disease Type C, Smith-Lemli-Opitz and Alzheimer's disease. Cholesterol plays a critical role in brain development, synaptogenesis, neuronal activity, and neuron survival. In the CNS, the BBB is not permeable to cholesterol and the brain sterol pool comes from in situ synthesis, mostly from astrocytes. (Boussicault, Lydie et al. “CYP46A1, the rate-limiting enzyme for cholesterol degradation, is neuroprotective in Huntington's disease.” Brain : a journal of neurology vol. 139, Pt 3 (2016): 953-70. doi:10.1093/brain/awv384).

CYP46A1 protein levels were decreased in the putamen, but not cerebral cortex samples, of post-mortem Huntington's disease patients when compared to controls. Cyp46A1 mRNA and CYP46A1 protein levels were also decreased in the striatum of the R6/2 Huntington's disease. Study design have shown that CYP46A1 knockout resulted in motor dysfunction and neuronal death due to the buildup of excess cholesterol and inability to metabolize sufficient amount for expulsion from the BBB. (Boussicault, 2016).

Other studies have expanded on the impact of increasing CYP46A1 expression and the protective effects against the development and progression of Huntington's Disease. In particular, CYP46A1 overexpression improves axonal BDNF vesicles trafficking in Huntington' disease cortical neurons and postsynaptic dynamics in Huntington' disease striatal neurons. (Kacher, Radhia & Lamazière, Antonin & Heck, Nicolas & Kappes, Vincent & Mounier, Coline & Despres, Gaëtan & Dembitskaya, Yulia & Perrin, Elodie & Christaller, Wilhelm & Nair, Satish & Messent, Valerie & Cartier, Nathalie & Vanhoutte, Peter & Venance, Laurent & Saudou, Frédéric & Neri, Christian & Caboche, Jocelyne & Betuing, Sandrine. (2019). CYP46A1 gene therapy deciphers the role of brain cholesterol metabolism in Huntington's disease. Brain : a journal of neurology. 142. 10.1093/brain/awz174).

Increasing expression of CYP46A1, and thereby increasing the enzymatic of CYP46A1 is protective against the development of neurodegenerative diseases caused by or associated with the buildup or inability to clear excess cholesterol in the brain. Furthermore, the beneficial impact on BDNF by increased CYP46A1 expands the capability for increasing expression of CYP46a1 on mental disorders and diseases associated with decreased BDNF activity. For example, studies have shown that increased CYP46A1 activity significantly improved cognative ability and spatial memory. Overexpression of CYP46A1 in a mouse model with increased tendency to accumulate b-amyloid in the brain was shown to decrease amyloid deposition and to improve cognition. (Is It Possible to Improve Memory Function by Upregulation of the Cholesterol 24S-Hydroxylase (CYP46A1) in the Brain?, Maioli S, Bavner A, Ali Z, Heverin M, Ismail M A M, et al. (2013) Is It Possible to Improve Memory Function by Upregulation of the Cholesterol 24S-Hydroxylase (CYP46A1) in the Brain?. PLOS ONE 8(7): e68534. https://doi.org/10.1371/journal.pone.0068534).

### Chronic Liver Diseases

Chronic liver disease is marked by the gradual destruction of liver tissue over time. Chronic liver disease is another factor that has been reported to impair P450 drug metabolism in patients (Villeneuve and Pichette, 2004)., hepatitis B and C, alcoholic liver disease, and cirrhosis (George et al., 1995a; Yang et al., 2003; Tsunedomi et al., 2005; Frye et al., 2006; Li et al., 2006b). (Fisher, 2009).

CYP450 activity has been shown to be decreased in primary biliary cirrhosis, alcoholic steatohepatitis, and cirrhotic patients as seen by reduced clearance of known substrates antipyrine, theophylline, and caffeine (Bechtel et al., 2000; Lelouët et al., 2001; Villeneuve and Pichette, 2004). (Fisher, 2009). Where the decrease in CYP expression and activity is caused by the disease, the decreased expression is generally associated with disease progression and disruption of healthy metabolic activity. Increasing CYP450 enzyme activity through increased expression of the encoding genes treats the dysfunction inhibition of the enzymatic activity by the physiological impact decreasing the same.

For example, CYP2C19 has been shown to be susceptible to decreased expression to the presence of liver diseases such as hepatocellular carcinoma, hepatitis C, and chronic hepatitis and cirrhosis (Ohnishi et al., 2005; Frye et al., 2006). In addition, it has also been shown to be affected earlier than the other important drug-metabolizing P450s (Villeneuve and Pichette, 2004). Results in the current study support these observations as the protein expression (p=0.010) and enzymatic activity (p=0.05) showed statistically significant decreases with progressive states of NAFLD. (Fisher, 2009). Increasing and positively regulating the expression of CYP450 genes such as CYP2C19, improves their function and maintains a healthy state of metabolism preventing and inhibiting the development and progression of chronic liver diseases.

### Non-Alcoholic Fatty Liver Disease

Nonalcoholic fatty liver disease (NAFLD) is generally characterized by excessive fat in the liver. In particular, NAFLD is identified by lipid accumulation within hepatocytes (steatosis) in the absence of excess alcohol consumption. It is the most common liver disease in the western world, affecting one third of the general adult population with particularly high prevalence in obesity and type 2 diabetes. NAFLD is a disease continuum originating with simple hepatic steatosis that can progress to non-alcoholic steatohepatitis (NASH) with fibrosis and potentially cirrhosis, which places patients at risk for hepatocellular carcinoma. (Woolsey, Sarah J., “Cytochrome P450 3A4 Expression and Regulation in Non-Alcoholic Fatty Liver Disease” (2015). Electronic Thesis and Dissertation Repository. 3355. https://ir.lib.uwo.ca/etd/3355).

In the early stages of NAFLD, an imbalance between uptake and export of lipids by hepatocytes leads to lipid accumulation within the liver. Increased hepatic saturated fatty acids and cholesterol activate toll-like receptors (TLRs) that drive activation of nuclear factor-κB (NF-κB)-mediated inflammatory responses. Sustained activation of the hepatic inflammatory response drives macrophage infiltration into the liver, ultimately causing fibrosis and hepatic injury. Consistent with this pathological progression of NAFLD/NASH, the high-fat/high-cholesterol “atherogenic” diet model of steatohepatitis induces dyslipidemia, hepatic inflammation, and fibrosis, through an innate immune-mediated mechanism. (The Cytochrome P450 Epoxygenase Pathway Regulates the Hepatic Inflammatory Response in Fatty Liver Disease. Schuck RN, Zha W, Edin ML, Gruzdev A, Vendrov KC, et al. (2014) The Cytochrome P450 Epoxygenase Pathway Regulates the Hepatic Inflammatory Response in Fatty Liver Disease. PLOS ONE 9(10): e110162. https://doi.org/10.1371/journal.pone.0110162).

Arachidonic acid is metabolized by and cytochromes P450 to biologically active eicosanoids, which are critical regulators of numerous biological processes including inflammation. CYP enzymes are abundantly expressed in the liver. CYP enzymes from the CYP2C and CYP2J subfamilies metabolize arachidonic acid to biologically active epoxyeicosatrienoic acids (EETs). However, EETs are rapidly hydrolyzed by soluble epoxide hydrolase (sEH, Ephx2) to their corresponding dihydroxyeicosatrienoic acid (DHETs), which are generally less biologically active. Studies show that acute, lipopolysaccharide (LPS)-evoked activation of the innate immune response suppresses hepatic CYP expression and EET biosynthesis. Moreover, increased endothelial EET biosynthesis, or decreased global sEH-mediated EET hydrolysis, attenuates NF-κB activation and the acute vascular and systemic inflammatory response to LPS. (Schuck, 2014). Hepatic EET biosynthesis is suppressed in response to activation of the innate immune system, and potentiation of the CYP pathway attenuates innate immune-dependent acute inflammatory responses. Atherogenic diet administration evoked a marked suppression of hepatic Cyp2c29, Cyp2c50, Cyp2c55, and Cyp2j5 expression, the most abundant CYP epoxygenases in mouse liver. (Id.). Studies have demonstrated that LPS-induced inflammation suppresses hepatic CYP epoxygenase expression in vivo. In addition, inflammatory cytokines including IL-1, IL-6 and TNFα suppress CYP expression in hepatocytes, and cytokine-mediated CYP suppression is dependent on NF-κB activation. (Schuck, 2014).

The inhibition of the CYP enxymes and their metabolic activity, as described above, is responsible for the development, progression, and increased damage caused by NAFLD. In particular, the expression and activity of CYP1A2, CYP2C19, CYP2D6, and CYP3A4 has been shown to be decreased and the decrease is associated with increasing severity of NAFLD. (Fisher, Craig D et al. “Hepatic cytochrome P450 enzyme alterations in humans with progressive stages of nonalcoholic fatty liver disease.” Drug metabolism and disposition: the biological fate of chemicals vol. 37,10 (2009): 2087-94. doi:10.1124/dmd.109.027466). A healthy state of metabolism and the prevention of NAFLD highlights the crucial need to regulate and promote CYP450 expression and activity to prevent NAFLD and reduce the severity of the damage caused.

### Cirrhosis

Cirrhosis, a chronic liver disease, is a condition in which your liver is scarred and permanently damaged. Scar tissue replaces healthy liver tissue and prevents your liver from working normally. Cirrhosis progression results in liver failure and death. Cirrhosis is the common endpoint of many hepatic diseases and represents a significant risk for liver failure and hepatocellular carcinoma. The development of cirrhosis is a continuous process from inflammation to fibrosis.

Studies have shown increased risk and incidence of cirrhosis associated with inhibited or decreased activity of the CYP450 gene family. For example, CYP 3A was found to be decreased in non-cholestatic cirrhosis due to hepatocellular diseases, and CYP 2E1 was significantly reduced in cholestatic cirrhosis (Dietrich CG, Götze O, Geier A. Molecular changes in hepatic metabolism and transport in cirrhosis and their functional importance. World Journal of Gastroenterology. 2016 Jan; 22(1): 72-88. DOI: 10.3748/wjg.v22.i1.72).

### Hepatocellular Carcinoma

Hepatocellular carcinoma (HCC) is a primary malignancy of the liver] that occurs predominantly in patients with underlying chronic liver disease and cirrhosis. However, up to 25% of patients have no history of cirrhosis or risk factors for it. The incidence of HCC has been rising worldwide over the last 20 years and is expected to increase until 2030 in some countries, including the United States. The incidence of HCC is highest in Asia and Africa, where the endemic high prevalence of hepatitis B and hepatitis C strongly predisposes to the development of chronic liver disease and subsequent development of HCC. (Luca Cicalese, Hepatocellular Carcinoma (HCC), https://emedicine.medscape.com/article/197319-overview, Jan. 31, 2021).

The protective effect of increasing CYP450 expression is multifaceted considering the development and progression of cancers such as hepatocellular carcinoma. As CYP450 enzymes are protective against several risk factors known to cause HCC, it is clear that the increase in CYP450 expression by a nitroxide antioxidant exerts a preventative effect against HCC development and progress. Furthermore, CYP450 expression and activity is directly associated with the prevention of tumorigenesis and HCC progression independent of the risk factors described here. CYP450 expression levels are greatly disrupted by the tumorigenic process. (Yan, Tongmeng et al. “Severely Impaired and Dysregulated Cytochrome P450 Expression and Activities in Hepatocellular Carcinoma: Implications for Personalized Treatment in Patients.” Molecular cancer therapeutics vol. 14,12 (2015): 2874-86. doi:10.1158/1535-7163.MCT-15-0274). In particular, hepatocellular carcinoma. Liver cancer is the fifth most common cancer and the second most frequent cause of death from cancer worldwide. Hepatocellular carcinoma (HCC) represents more than 90% of primary liver cancers and is a major global health problem. Chronic infection with hepatitis B virus (HBV) is the most clearly established risk factor for HCC, and approximately 54% of cases can be attributed to HBV infection. (Yan, 2015).

### Methods for Counteracting Age-Related Decrease in Gene Expression or Treating a Condition Related to Aging

Some embodiments disclosed herein provide methods for counteracting age-related decrease in gene expression or treating an age-related disease, comprising identifying a human subject over the age of 35 and having a decreased expression level of one or more genes associated with the cytochrome p450 family or an age-related disease; and administering to the human subject an effective amount of a nitroxide antioxidant. In some embodiments, the methods comprise determining the expression level of one or more genes associated with the cytochrome p450 family. However, this may not be necessary in some instances, such as where a decreased expression level of one or more genes associated with the cytochrome p450 family can be inferred from the human subject's age, family history, health conditions, medical history, habits, or a combination thereof. In some embodiments, the methods disclosed herein may be used to treat a human subject shows no symptoms of an age-related disease, but is at risk of having an age-related disease. Exemplary risk factors for an age-related disease include, but are not limited to, age, family history, health conditions, medical history, habits, or a combination thereof. In some embodiments, risk factors for an age-related disease comprise a decreased expression level of one or more genes associated with the cytochrome p450 family.

In some embodiments, administering to the human subject an effective amount of the nitroxide antioxidant results in an increased expression level of a gene, for example a gene associated with the cytochrome p450 family. The gene associated with the cytochrome p450 family can be Cyp2c29, Cyp3a25, Cyp3a11, Cyp2j5, Cyp2c50, Cyp2c55, Cyp2d9, Cyp2e1, Cyp2b9, Cyp3a13, Cyp4f15, Cyp2c70, Cyp46a1, Cyp27a1, or Cyp4v3. The treatment of the human subject with the effective amount of the nitroxide antioxidant results in an increased expression level of the gene. For example, the treatment results in increased expression levels of Cyp2c29, Cyp3a25, Cyp3a11, Cyp2j5, Cyp2c50, Cyp2c55, Cyp2d9, Cyp2e1, Cyp2b9, Cyp3a13, Cyp4f15, Cyp2c70, Cyp46a1, Cyp27a1, Cyp4v3, or any combination thereof. The increased expression levels of Cyp2c29, Cyp3a25, Cyp3a11, Cyp2j5, Cyp2c50, Cyp2c55, Cyp2d9, Cyp2e1, Cyp2b9, Cyp3a13, Cyp4f15, Cyp2c70, Cyp46a1, Cyp27a1, Cyp4v3, or any combination thereof, can increase the level of apoptosis. The increased level of apoptosis results in a decrease in or disappearance of signs and symptoms of an age-related disease associated with decreased apoptosis, including the curing of the age-related disease. In some embodiments, the increased expression levels of Cyp2c29, Cyp3a25, Cyp3a11, Cyp2j5, Cyp2c50, Cyp2c55, Cyp2d9, Cyp2e1, Cyp2b9, Cyp3a13, Cyp4f15, Cyp2c70, Cyp46a1, Cyp27a1, Cyp4v3, or any combination thereof, can decrease the level of apoptosis. The decreased level of apoptosis results in a decrease in or disappearance of signs and symptoms of the age-related disease associated with increased apoptosis, including the curing of the disease associated with age-related disease associated with increased apoptosis.

In some embodiments, the levels of Cyp2c29, Cyp3a25, Cyp3a11, Cyp2j5, Cyp2c50, Cyp2c55, Cyp2d9, Cyp2e1, Cyp2b9, Cyp3a13, Cyp4f15, Cyp2c70, Cyp46a1, Cyp27a1, Cyp4v3, or any combination thereof in the connective tissue, muscle tissue, nervous tissue, or epithelial tissue may change after the nitroxide antioxidant is administered. Non-limiting examples of the connective tissue include dense connective tissue, loose connective tissue, reticular connective tissue, adipose tissue, cartilage, bone, and extracellular matrix. Non-limiting examples of the muscle tissue includes smooth muscle tissue, cardiac muscle tissue, and skeletal muscle tissue. Non-limiting examples of the nervous tissue include neural tissue of the central nervous system, neural tissue of the peripheral nervous system, the brain, spinal cord, cranial nerves, spinal nerves, and motor neurons. Non-limiting examples of the epithelial tissue include squamous epithelium, cuboidal epithelium, columnar epithelium, glandular epithelium, ciliated epithelium, and skin.

Some embodiments disclosed herein provide methods for treating a disease related to aging in a human subject in need thereof, comprising identifying a human subject over the age of 35 and having an age-related disease and having a decreased expression level of a gene associated with the cytochrome p450 family; and administering to the human subject an effective amount of a nitroxide antioxidant. Some embodiments disclosed herein provide methods for treating an individual having or at risk of developing a condition due to aging, comprising: identifying an individual over the age of 35; and administering to the individual an effective amount of a nitroxide antioxidant, whereby the expression level of the gene associated with the cytochrome p450 family is increased.

Non-limiting examples of age-related diseases include cancer, rheumatoid/osteoid arthritis, systemic lupus erythematosus (SLE), inflammatory bowel disease, Alzheimer's disease, multiple sclerosis, atherosclerosis, cardiovascular disease, cataracts, dementia, osteoporosis, type 2 diabetes, hypertension.

### Xenobiotic-Associated Toxicities and Addiction

A direct association between impaired xenobiotic metabolism and increased or unexpected toxicity is attributable to underexpressed or inhibitied cytochrome p450 enzymes. For example, recreational and medicinal drugs have unintended negative consequences when they are poorly metabolized leading to accumulation of the drug, prolonged impact of the drug effects. Individuals who experience these negative side effects and potentially lethal toxicities benefit from a responsive administration of Tempol to increase an impaired cyp450 enzymatic action to metabolize the substrate drug compound.

Methadone has become more widely prescribed for pain control in the USA since 1997 when new clinical guidelines for pain management were introduce. Indications for prescribing methadone now include pain relief (for severe, malignant and postoperative pain), detoxification of narcotic addiction and temporary maintenance treatment of narcotic addiction. Methadone is an efficacious method of treatment for heroin addiction because of high bioavailability, long elimination half-life, lack of detrimental behavior modification and the availability of the antagonist naloxone as an antidote. However, selection of the appropriate methadone dose is difficult because a given dose results in a wide range of interindividual pharmacokinetics, increasing the likelihood of an adverse drug reaction (ADR). Much of the described interindividual variability in response to methadone can be explained by polymorphisms in a variety of proteins, including the CYP450 enzymes responsible for methadone metabolism. The principal metabolite of methadone is 2-ethylidene-1,5-dimethyl-3,3-diphenylpyrrolidine (EDDP), which is formed during first-pass metabolism through an N-demethylation reaction followed by spontaneous cyclization. CYP involvement in methadone metabolism is stereoselective. CYP3A4 is thought to metabolize both enantiomers, but is the primary CYP isozyme mediating R-methadone metabolism. A 60-85% decrease in EDDP formation following chemical or monoclonal antibody inhibition of the CYP3A4 was observed in human liver microsomes. CYP2D6 also metabolizes the R-enantiomer, but the contribution by this isozyme is minor. The (S)-methadone enantiomer is mostly metabolized by CYP2B6. Drugs with agonist activity at the μ- or δ-opioid receptors can cause respiratory depression, which is the most common mechanism by which methadone causes death. Thus, CYP variants could cause or contribute to the poor methadone metabolizer phenotype leading to an increased likelihood for fatal methadone toxicity. (Richards-Waugh, Lauren L et al. “Fatal methadone toxicity: potential role of CYP3A4 genetic polymorphism.” Journal of analytical toxicology vol. 38, 8 (2014): 541-7. doi: 10.1093/jat/bku091).

CYP enzymes play an essential role in maintaining the levels of bioactive molecules within normal ranges. These enzymes modulate the metabolism of endogenous neurochemicals, such as neurosteroids, dopamine, serotonin, melatonin, anandamide, and exogenous substances, including psychotropics, drugs of abuse, neurotoxins, and carcinogens. The role of these enzymes is not restricted to xenobiotic-induced neurotoxicity, but they are also involved in brain physiology. Nitroxide antioxidants, such as Tempol, are able to pass the blood brain barrier and confer beneficial impact on the CYP450 encoding genes within the brain. CYP enzymes are differentially expressed in individual regions of the brain and in specific neuronal and glial cells. The highest CYP content is found in the brain stem (the structure containing cell bodies of dopamine, noradrenaline, and serotonin neurons), cerebellum (Purkinje cells in particular; the structure engaged in motor control, cognitive and emotional functions), hippocampus (the structure involved in learning and memory), ventral striatum containing the nucleus accumbens (the structure engaged in cognitive functions, reward, and addiction), and dorsal striatum (the structure involved in motor functions) (Dutheil et al. 2008). Drug metabolizing CYPs, 1A1, 1A2, 2A6, 2B1, 2E1, and 3A4, occur predominantly in neurons (Bhagwat et al. 2000; Singh et al. 2012), whereas CYP1B1 and CYP2D6 are expressed in both neurons and glial cells (Gilham et al. 1997; Muskhelishvili et al. 2001; Miksys et al. 2003). (Wojciech Kuban & Wladyslawa Anna Daniel (2021) Cytochrome P450 expression and regulation in the brain, Drug Metabolism Reviews, 53: 1, 1-29, DOI: 10.1080/03602532.2020.1858856, the disclosure of which is incorporated herein in its entirety).

Accordingly, modulation of CYP450 enzymes by a nitroxide antioxidant, such as Tempol, is essential to address adverse drug reactions and associated toxicities in individuals having a suppressed expression of one or more CYP450 encoding genes. Additionally, regulation of the CYP450 gene family treats and prevents these ADRs well as the underlying psychological drivers associated with addiction.

### Methods for Increasing Expression Level of a Gene

Some embodiments disclosed herein provide methods for increasing the expression level of a gene in a human subject in need thereof, comprising identifying a human subject having a decreased expression level of a gene associated with the cytochrome p450 family; and administering to the human subject an effective amount of a nitroxide antioxidant. Some embodiments disclosed herein provide methods for treating a disease associated with a decreased apoptosis in a patient in need thereof, comprising identifying a human subject having a decreased expression level of a gene associated with the cytochrome p450 family; and administering to the human subject an effective amount of a nitroxide antioxidant. The decreased expression level may be age-related, or disease related. In some embodiments, the disease may be cancer, rheumatoid/osteoid arthritis, systemic lupus erythematosus (SLE), inflammatory bowel disease, Alzheimer's disease, multiple sclerosis, atherosclerosis, cardiovascular disease, cataracts, dementia, osteoporosis, type 2 diabetes, hypertension, or any combination thereof. Some embodiments disclosed herein provide methods for treating an individual in need thereof, comprising identifying a human subject over the age of 35 in need of an increased expression level of a gene associated with the cytochrome p450 family; and administering to the human subject an effective amount of a nitroxide antioxidant. In some embodiments, the methods comprise determining the expression level of one or more genes associated with the cytochrome p450 family. However, this may not be necessary in some instances, such as where a decreased expression level of one or more genes associated with the cytochrome p450 family can be inferred from the human subject's age, family history, health conditions, medical history, habits, or a combination thereof. In some embodiments, the methods disclosed herein may be used to treat a human subject shows no symptoms of a disease associated with a decreased apoptosis, but is at risk of having a disease associated with a decreased apoptosis. Exemplary risk factors for a disease associated with a decreased apoptosis include, but are not limited to, age, family history, health conditions, medical history, habits, or a combination thereof.

In some embodiments, administering to the human subject an effective amount of the nitroxide antioxidant results in an increased expression level of a gene, for example a gene associated with the cytochrome p450 family. The gene associated with the cytochrome p450 family can be Cyp2c29, Cyp3a25, Cyp3a11, Cyp2j5, Cyp2c50, Cyp2c55, Cyp2d9, Cyp2e1, Cyp2b9, Cyp3a13, Cyp4f15, Cyp2c70, Cyp46a1, Cyp27a1, or Cyp4v3. The treatment of the human subject with the effective amount of the nitroxide antioxidant results in an increased expression level of the gene. For example, the treatment can increase the expression levels of Cyp2c29, Cyp3a25, Cyp3a11, Cyp2j5, Cyp2c50, Cyp2c55, Cyp2d9, Cyp2e1, Cyp2b9, Cyp3a13, Cyp4f15, Cyp2c70, Cyp46a1, Cyp27a1, Cyp4v3, or any combination thereof. The increased expression of the gene counteracts the decrease in the expression level of the gene.

### Methods for Preventing Cancer

Some embodiments disclosed herein provide methods for preventing cancer in a human subject, comprising identifying a human subject in need of an increased expression level of a gene associated with the cytochrome p450 family; and administering to the human subject an effective amount of a nitroxide antioxidant. In some embodiments, the methods disclosed herein may be used to prevent cancer development in a human subject showing no symptoms of cancer, but is at risk of having cancer. Exemplary risk factors for cancer include, but are not limited to, age, family history, health conditions, medical history, habits, or a combination thereof. In some embodiments, risk factors for cancer comprise a decreased expression level of one or more genes associated with the cytochrome p450 family.

In some embodiments cancer development is mediated by unregulated cell growth and dysfunction in proto oncogenes. Xenobiotics results in genotoxicity and mutations in DNA sequences involved in the transcription or translation of genes involved in cellular function including cell growth and development. For example, proto oncogenes are susceptible to mutation through interaction between xenobiotic factors and corresponding DNA sequences. Also known as genotoxic xenobiotics, such xenobiotic substances and factors contribute to the unregulated cell growth and development attributed to carcinogenesis. The accumulation of genetic damage in the forms of activated proto-oncogenes and inactivated tumor-suppressor genes is the driving force in the evolution of a normal cell to a malignant cell. Activation of the ras gene is an early event--probably the “initiating” step--in the development of many chemical-induced rodent tumors. ras Oncogenes are observed in more human tumors and at a higher frequency than any other oncogene, and activation of the proto-oncogene occurs at various stages of the carcinogenic process. (Anderson, M. W., Reynolds, S. H., You, M., & Maronpot, R. M. (1992). Role of proto-oncogene activation in carcinogenesis. Environmental Health Perspectives, 98, 13-24, the disclosure of which is incorporated herein in its entirety).

Chemical, environmental, endogenous and exogenous factors are or result in the presence of xenobiotics. Increased xenobiotic concentrations correlate with increased occurrence of oncogenes such as ras. For example, overexpression of the ras oncogene in plasma was found in the samples taken during winter, suggesting a strong influence of complex exposure caused by domestic coal heating. (Silins, I., & Hogberg, J. (2011). Combined Toxic Exposures and Human Health: Biomarkers of Exposure and Effect. International Journal of Environmental Research and Public Health, 8(3), 629-647. http://doi.org/10.3390/ijerph8030629, the disclosure of which is incorporated herein in its entirety). Another example of xenobiotic associated carcinogenesis is seen in the mechanisms and toxicological consequences of oxidative stress triggered by metals and dietary or environmental pollutants in general. Besides causing DNA damage, ROS further induces multiple intracellular signaling pathways, notably NF-κB, JNK/SAPK/p38, as well as Erk/MAPK. These signaling routes can lead to transcriptional induction of target genes that could promote proliferation or confer apoptosis resistance to exposed cells. (Henkler, F., Brinkmann, J., & Luch, A. (2010). The Role of Oxidative Stress in Carcinogenesis Induced by Metals and Xenobiotics. Cancers, 2(2), 376-396. http://doi.org/10.3390/cancers2020376, the disclosure of which is incorporated herein in its entirety). Therefore, the regulation and increase of GST activity and the corresponding increase in glutathione activity in addressing xenobiotics, by administration of a nitroxide antioxidant, prevents cancer.

Non-limiting examples of the methods for identifying a human subject at risk for cancer include colonoscopy; sigmoidoscopy; and high-sensitivity fecal occult blood tests. In some embodiments, methods for identifying a human subject having a cancer include low-dose helical computed tomography; mammography; and pap test and human papillomavirus (HPV) testing. In some embodiments, methods for identifying a human subject having a cancer include alpha-fetoprotein blood test; breast magnetic resonance imaging (MRI); CA-125 test; clinical breast exams and regular breast self-exams; prostate-specific antigen (PSA) testing; skin exams; transvaginal ultrasound; and virtual colonoscopy. In some embodiments, methods for identifying a human subject having a cancer include barium enema; biopsy; bone marrow aspiration and biopsy; bone scan; breast MRI for early detection of breast cancer; breast MRI; colonoscopy; computed tomography (CT) scan; digital rectal exam (DRE); blood and platelets testing; bone marrow testing; umbilical cord blood testing; electrocardiogram (EKG) and echocardiogram; endoscopic techniques; fecal occult blood tests; magnetic resonance imaging (MRI); mammography; multi gated acquisition (MUGA) scan; papanicolaou (pap) test; positron emission tomography and computed tomography (PET-CT) scan; sigmoidoscopy; tumor marker tests; ultrasound; upper endoscopy. In some embodiments, methods for identifying a human subject having a cancer include DNA sequencing; detecting presence of single nucleotide polymorphism (SNIP); and detecting the presence of certain protein markers.

In some embodiments, administering to the human subject an effective amount of the nitroxide antioxidant results in an increased expression level of a gene, for example a gene associated with the cytochrome p450 family. The gene associated with the cytochrome p450 family can be Cyp2c29, Cyp3a25, Cyp3a11, Cyp2j5, Cyp2c50, Cyp2c55, Cyp2d9, Cyp2e1, Cyp2b9, Cyp3a13, Cyp4f15, Cyp2c70, Cyp46a1, Cyp27a1, or Cyp4v3. The treatment of the human subject with the effective amount of the nitroxide antioxidant results in an increased expression of the gene. For example, the treatment results in increased expression levels of Cyp2c29, Cyp3a25, Cyp3a11, Cyp2j5, Cyp2c50, Cyp2c55, Cyp2d9, Cyp2e1, Cyp2b9, Cyp3a13, Cyp4f15, Cyp2c70, Cyp46a1, Cyp27a1, Cyp4v3, or any combination thereof. The increased expression level of the gene can increase the level of GST activity. For example, increased capacity to remove xenobiotics and genotoxic xenobiotics. The increased level of GST activity results in prevention of proto oncogene mutations to oncogenes and prevention of associated cancer.

### Methods for Treating Cancer

Some embodiments disclosed herein provide methods for treating cancer in a human subject in need thereof, comprising identifying a human subject having a cancer and in need of an increased expression level of a gene associated with the cytochrome p450 family; and administering to the human subject an effective amount of a nitroxide antioxidant. In some embodiments, the methods disclosed herein may be used to treat a human subject shows no symptoms of cancer, but is at risk of having cancer. Exemplary risk factors for cancer include, but are not limited to, age, family history, health conditions, medical history, habits, or a combination thereof. In some embodiments, risk factors for cancer comprise a decreased expression level of one or more genes associated with the cytochrome p450 family.

Non-limiting examples of the methods for identifying a human subject having a cancer include colonoscopy; sigmoidoscopy; and high-sensitivity fecal occult blood tests. In some embodiments, methods for identifying a human subject having a cancer include low-dose helical computed tomography; mammography; and pap test and human papillomavirus (HPV) testing. In some embodiments, methods for identifying a human subject having a cancer include alpha-fetoprotein blood test; breast magnetic resonance imaging (MRI); CA-125 test; clinical breast exams and regular breast self-exams; prostate-specific antigen (PSA) testing; skin exams; transvaginal ultrasound; and virtual colonoscopy. In some embodiments, methods for identifying a human subject having a cancer include barium enema; biopsy; bone marrow aspiration and biopsy; bone scan; breast MRI for early detection of breast cancer; breast MRI; colonoscopy; computed tomography (CT) scan; digital rectal exam (DRE); blood and platelets testing; bone marrow testing; umbilical cord blood testing; electrocardiogram (EKG) and echocardiogram; endoscopic techniques; fecal occult blood tests; magnetic resonance imaging (MRI); mammography; multi gated acquisition (MUGA) scan; papanicolaou (pap) test; positron emission tomography and computed tomography (PET-CT) scan; sigmoidoscopy; tumor marker tests; ultrasound; upper endoscopy. In some embodiments, methods for identifying a human subject having a cancer include DNA sequencing; detecting presence of single nucleotide polymorphism (SNIP); and detecting the presence of certain protein markers.

In some embodiments, administering to the human subject an effective amount of the nitroxide antioxidant results in an increased expression level of a gene, for example a gene associated with the cytochrome p450 family. The gene associated with the cytochrome p450 family can be Cyp2c29, Cyp3a25, Cyp3a11, Cyp2j5, Cyp2c50, Cyp2c55, Cyp2d9, Cyp2e1, Cyp2b9, Cyp3a13, Cyp4f15, Cyp2c70, Cyp46a1, Cyp27a1, or Cyp4v3. The treatment of the human subject with the effective amount of the nitroxide antioxidant results in an increased expression of the gene. For example, the treatment results in increased expression levels of Cyp2c29, Cyp3a25, Cyp3a11, Cyp2j5, Cyp2c50, Cyp2c55, Cyp2d9, Cyp2e1, Cyp2b9, Cyp3a13, Cyp4f15, Cyp2c70, Cyp46a1, Cyp27a1, Cyp4v3, or any combination thereof. The increased expression level of the gene can increase the level of apoptosis. The increased level of apoptosis results in a decrease in or disappearance of signs and symptoms of the cancer, including the curing of the cancer.

Non-limiting examples of cancer include bladder and other urothelial cancers; breast cancer; cervical cancer; colorectal cancer; endometrial cancer; endometrial cancer; esophageal cancer; liver (hepatocellular) cancer; lung cancer; neuroblastoma cancer; oral cavity and oropharyngeal cancer; ovarian, fallopian tube, and primary peritoneal cancer; prostate cancer; skin cancer; stomach (gastric) cancer; and testicular cancer.

Non-limiting examples of cancer include acute lymphoblastic leukemia, adult; acute myeloid leukemia, adult; adrenocortical carcinoma; aids-related lymphoma; anal cancer; bile duct cancer; bladder cancer; brain tumors, adult; breast cancer; breast cancer and pregnancy; breast cancer, male; carcinoid tumors, gastrointestinal; carcinoma of unknown primary; cervical cancer; chronic lymphocytic leukemia; chronic myelogenous leukemia; chronic myeloproliferative neoplasms; cns lymphoma, primary; colon cancer; endometrial cancer; esophageal cancer; extragonadal germ cell tumors; fallopian tube cancer; gallbladder cancer; gastric cancer; gastrointestinal carcinoid tumors; gastrointestinal stromal tumors; germ cell tumors, extragonadal; germ cell tumors, ovarian; gestational trophoblastic disease; hairy cell leukemia; hepatocellular (liver) cancer, adult primary; histiocytosis, langerhans cell; hodgkin lymphoma, adult; hypopharyngeal cancer; intraocular (eye) melanoma; islet cell tumors, pancreatic neuroendocrine tumors; kaposi sarcoma; kidney (renal cell) cancer; kidney (renal pelvis and ureter, transitional cell) cancer; langerhans cell histiocytosis; laryngeal cancer; leukemia, adult acute lymphoblastic; leukemia, adult acute myeloid; leukemia, chronic lymphocytic; leukemia, chronic myelogenous; leukemia, hairy cell; lip and oral cavity cancer; liver cancer, adult primary; lung cancer, non-small cell; lung cancer, small cell; lymphoma, adult Hodgkin; lymphoma, adult non-hodgkin; lymphoma, aids-related; lymphoma, primary cns; malignant mesothelioma; melanoma; melanoma, intraocular (eye); merkel cell carcinoma; metastatic squamous neck cancer with occult primary; multiple myeloma and other plasma cell neoplasms; mycosis fungoides and the sézary syndrome; myelodysplastic syndromes; myelodysplastic/myeloproliferative neoplasms; myeloproliferative neoplasms, chronic; paranasal sinus and nasal cavity cancer; nasopharyngeal cancer; neck cancer with occult primary, metastatic squamous; non-hodgkin lymphoma, adult; non-small cell lung cancer; oral cavity cancer, lip oropharyngeal cancer; ovarian epithelial cancer; ovarian germ cell tumors; ovarian low malignant potential tumors; pancreatic cancer; pancreatic neuroendocrine tumors (islet cell tumors); pheochromocytoma and paraganglioma; paranasal sinus and nasal cavity cancer; parathyroid cancer; penile cancer; pheochromocytoma and paraganglioma; pituitary tumors; plasma cell neoplasms, multiple myeloma and other; breast cancer and pregnancy; primary peritoneal cancer; prostate cancer; rectal cancer; renal cell cancer; transitional cell renal pelvis and ureter; salivary gland cancer; sarcoma, Kaposi; sarcoma, soft tissue, adult; sarcoma, uterine; mycosis fungoides and the sézary syndrome; skin cancer, melanoma; skin cancer, nonmelanoma; small cell lung cancer; small intestine cancer; stomach (gastric) cancer; testicular cancer; thymoma and thymic carcinoma; thyroid cancer; transitional cell cancer of the renal pelvis and ureter; trophoblastic disease, gestational; carcinoma of unknown primary; urethral cancer; uterine cancer, endometrial; uterine sarcoma; vaginal cancer; and vulvar cancer.

In some embodiments, non-limiting examples of cancer include, but are not limited to, hematologic and solid tumor types such as acoustic neuroma, acute leukemia, acute lymphoblastic leukemia, acute myelogenous leukemia (monocytic, myeloblastic, adenocarcinoma, angiosarcoma, astrocytoma, myelomonocytic and promyelocytic), acute t-cell leukemia, basal cell carcinoma, bile duct carcinoma, bladder cancer, brain cancer, breast cancer (including estrogen-receptor positive breast cancer), bronchogenic carcinoma, Burkitt's lymphoma, cervical cancer, chondrosarcoma, chordoma, choriocarcinoma, chronic leukemia, chronic lymphocytic leukemia, chronic myelocytic (granulocytic) leukemia, chronic myelogenous leukemia, colon cancer, colorectal cancer, craniopharyngioma, cystadenocarcinoma, dysproliferative changes (dysplasias and metaplasias), embryonal carcinoma, endometrial cancer, endotheliosarcoma, ependymoma, epithelial carcinoma, erythroleukemia, esophageal cancer, estrogen-receptor positive breast cancer, essential thrombocythemia, Ewing's tumor, fibrosarcoma, gastric carcinoma, germ cell testicular cancer, gestational trophobalstic disease, glioblastoma, head and neck cancer, heavy chain disease, hemangioblastoma, hepatoma, hepatocellular cancer, hormone insensitive prostate cancer, leiomyosarcoma, liposarcoma, lung cancer (including small cell lung cancer and non-small cell lung cancer), lymphangioendothelio-sarcoma, lymphangiosarcoma, lymphoblastic leukemia, lymphoma (lymphoma, including diffuse large B-cell lymphoma, follicular lymphoma, Hodgkin's lymphoma and non-Hodgkin's lymphoma), malignancies and hyPerproliferative disorders of the bladder, breast, colon, lung, ovaries, pancreas, prostate, skin and uterus, lymphoid malignancies of T-cell or B-cell origin, leukemia, medullary carcinoma, medulloblastoma, melanoma, meningioma, mesothelioma, multiple myeloma, myelogenous leukemia, myeloma, myxosarcoma, neuroblastoma, oligodendroglioma, oral cancer, osteogenic sarcoma, ovarian cancer, pancreatic cancer, papillary adenocarcinomas, papillary carcinoma, peripheral T-cell lymphoma, pinealoma, polycythemia vera, prostate cancer (including hormone-insensitive (refractory) prostate cancer), rectal cancer, renal cell carcinoma, retinoblastoma, rhabdomyosarcoma, sarcoma, sebaceous gland carcinoma, seminoma, skin cancer, small cell lung carcinoma, solid tumors (carcinomas and sarcomas), stomach cancer, squamous cell carcinoma, synovioma, sweat gland carcinoma, testicular cancer (including germ cell testicular cancer), thyroid cancer, Waldenstrom's macroglobulinemia, testicular tumors, uterine cancer, Wilms' tumor and the like.

Non-limiting examples of the cancer include acute lymphoblastic leukemia, childhood; acute myeloid leukemia/other myeloid malignancies, childhood; adrenocortical carcinoma, childhood; astrocytomas, childhood; atypical teratoid/rhabdoid tumor, childhood central nervous system; basal cell carcinoma, childhood; bladder cancer, childhood; bone, malignant fibrous histiocytoma of and osteosarcoma; brain and spinal cord tumors overview, childhood; brain stem glioma, childhood; (brain tumor), childhood astrocytomas; (brain tumor), childhood central nervous system atypical teratoid/rhabdoid tumor; (brain tumor), childhood central nervous system embryonal tumors; (brain tumor), childhood central nervous system germ cell tumors; (brain tumor), childhood craniopharyngioma; (brain tumor), childhood ependymoma; breast cancer, childhood; bronchial tumors, childhood; carcinoid tumors, childhood; carcinoma of unknown primary, childhood; cardiac (heart) tumors, childhood; central nervous system atypical teratoid/rhabdoid tumor, childhood; central nervous system embryonal tumors, childhood; central nervous system germ cell tumors, childhood; cervical cancer, childhood; chordoma, childhood; colorectal cancer, childhood; craniopharyngioma, childhood; effects, treatment for childhood cancer, late; embryonal tumors, central nervous system, childhood; ependymoma, childhood; esophageal tumors, childhood; esthesioneuroblastoma, childhood; ewing sarcoma; extracranial germ cell tumors, childhood; gastric (stomach) cancer, childhood; gastrointestinal stromal tumors, childhood; germ cell tumors, childhood central nervous system; germ cell tumors, childhood extracranial; glioma, childhood brain stem; head and neck cancer, childhood; heart tumors, childhood; hematopoietic cell transplantation, childhood; histiocytoma of bone, malignant fibrous and osteosarcoma; histiocytosis, langerhans cell; hodgkin lymphoma, childhood; kidney tumors of childhood, wilms tumor and other; langerhans cell histiocytosis; laryngeal cancer, childhood; late effects of treatment for childhood cancer; leukemia, childhood acute lymphoblastic; leukemia, childhood acute myeloid/other childhood myeloid malignancies; liver cancer, childhood; lung cancer, childhood; lymphoma, childhood Hodgkin; lymphoma, childhood non-Hodgkin; malignant fibrous histiocytoma of bone and osteosarcoma; melanoma, childhood; mesothelioma, childhood; midline tract carcinoma, childhood; multiple endocrine neoplasia, childhood; myeloid leukemia, childhood acute/other childhood myeloid malignancies; nasopharyngeal cancer, childhood; neuroblastoma, childhood; non-hodgkin lymphoma, childhood; oral cancer, childhood; osteosarcoma and malignant fibrous histiocytoma of bone; ovarian cancer, childhood; pancreatic cancer, childhood; papillomatosis, childhood; paraganglioma, childhood; pediatric supportive care; pheochromocytoma, childhood; pleuropulmonary blastoma, childhood; retinoblastoma; rhabdomyosarcoma, childhood; salivary gland cancer, childhood; sarcoma, childhood soft tissue; (sarcoma), ewing sarcoma; (sarcoma), osteosarcoma and malignant fibrous histiocytoma of bone; (sarcoma), childhood rhabdomyosarcoma; (sarcoma) childhood vascular tumors; skin cancer, childhood; spinal cord tumors overview, childhood brain and; squamous cell carcinoma (skin cancer), childhood; stomach (gastric) cancer, childhood; supportive care, pediatric; testicular cancer, childhood; thymoma and thymic carcinoma, childhood; thyroid tumors, childhood; transplantation, childhood hematopoietic; childhood carcinoma of unknown primary; unusual cancers of childhood; vaginal cancer, childhood; vascular tumors, childhood; and wilms tumor and other childhood kidney tumors.

Non-limiting examples of cancer include embryonal rhabdomyosarcoma, pediatric acute lymphoblastic leukemia, pediatric acute myelogenous leukemia, pediatric alveolar rhabdomyosarcoma, pediatric anaplastic ependymoma, pediatric anaplastic large cell lymphoma, pediatric anaplastic medulloblastoma, pediatric atypical teratoid/rhabdoid tumor of the central nervous system, pediatric biphenotypic acute leukemia, pediatric Burkitts lymphoma, pediatric cancers of Ewing's family of tumors such as primitive neuroectodermal rumors, pediatric diffuse anaplastic Wilm's tumor, pediatric favorable histology Wilm's tumor, pediatric glioblastoma, pediatric medulloblastoma, pediatric neuroblastoma, pediatric neuroblastoma-derived myelocytomatosis, pediatric pre-B-cell cancers (such as leukemia), pediatric psteosarcoma, pediatric rhabdoid kidney tumor, pediatric rhabdomyosarcoma, and pediatric T-cell cancers such as lymphoma and skin cancer.

### Methods for Preventing Autoimmune Diseases

Some embodiments disclosed herein provide methods for preventing an autoimmune disease in a human subject in need thereof, comprising identifying a human subject having an autoimmune disease and in need of an increased expression level of a gene associated with the cytochrome p450 family; and administering to the human subject an effective amount of a nitroxide antioxidant. In some embodiments, the methods disclosed herein may be used to prevent the development of an autoimmune disease in a human subject showing no symptoms of an autoimmune disease, but is at risk of having an autoimmune disease. Exemplary risk factors for an autoimmune disease include, but are not limited to, age, family history, health conditions, medical history, habits, or a combination thereof. In some embodiments, risk factors for an autoimmune disease comprise a decreased expression level of one or more genes associated with the cytochrome p450 family.

In some embodiments, Autoimmunity is the system of immune responses of an organism against its own healthy cells and tissues. Any disease that results from such an aberrant immune response is termed an “autoimmune disease”. Prominent examples include celiac disease, diabetes mellitus type 1, sarcoidosis, systemic lupus erythematosus (SLE), Sjogren's syndrome, eosinophilic granulomatosis with polyangiitis, Hashimoto's thyroiditis, Graves' disease, idiopathic thrombocytopenic purpura, Addison's disease, rheumatoid arthritis (RA), ankylosing spondylitis, polymyositis (PM), and dermatomyositis (DM). Autoimmune diseases are very often treated with steroids.

Toxicants, infections, epitope spreading, dysfunctions of immune homeostasis, and dietary components can all have an impact on the body's delicate immune recognition system. Although the precise etiology and pathogenesis of many autoimmune diseases are still unknown, it would appear from the collated studies that there are common mechanisms in the immunopathogenesis of multiple autoimmune reactivities. (Vojdani, A. A Potential Link between Environmental Triggers and Autoimmunity. Autoimmune Diseases, Volume 2014 (2014), Article ID 437231, 18 pages http://dx.doi.org/10.1155/2014/437231, the disclosure of which is incorporated herein in its entirety).

A number of clinical reports and experimental studies have shown that autoimmune responses and/or autoimmune diseases are induced in humans and laboratory animals by chronic exposure to various chemicals. (Bigazzi PE. Autoimmunity caused by xenobiotics. Toxicology. 1997 Apr. 11; 119(1): 1-21, the disclosure of which is incorporated herein in its entirety). A non-limiting example is seen in the correlation between acetaminophen and primary biliary cirrhosis, the serologic hallmark of primary biliary cirrhosis (PBC) is the presence of antimitochondrial autoantibodies (AMAs) directed against the E2 subunit of the pyruvate dehydrogenase complex (PDC-E2). The PBC-related autoepitope of PDC-E2 contains lipoic acid, and previous work has demonstrated that mimics of lipoic acid following immunization of mice lead to a PBC-like disease. Furthermore, approximately one-third of patients who have ingested excessive amounts of acetaminophen (paracetamol) develop AMA of the same specificity as patients with PBC. Quantitative structure—activity relationship (QSAR) data indicates that acetaminophen metabolites are particularly immunoreactive with AMA, and in genetically susceptible hosts, electrophilic modification of lipoic acid in PDC-E2 by acetaminophen or similar drugs can facilitate a loss of tolerance and lead to the development of PBC. (Leung, Patrick S. C. et al. Xenobiotics and autoimmunity: does acetaminophen cause primary biliary cirrhosis? Trends in Molecular Medicine, Volume 18, Issue 10, 577-582, the disclosure of which is incorporated herein in its entirety). Another example involves the development of hepatitis and associated environmental factors resulting in the development thereof. Exposure to certain xenobiotics such as trichloroethylene may disrupt certain mechanisms and promote autoimmune hepatitis. (Gilbert, K., Xenobiotic Exposure and Autoimmune Hepatitis. Hepatitis Research and Treatment Volume 2010 (2010), Article ID 248157, 10 pages http://dx.doi.org/10.1155/2010/248157, the disclosure of which is incorporated herein in its entirety). Therefore, the regulation and increase of GST activity and the corresponding increase in glutathione activity in addressing xenobiotics, by administration of a nitroxide antioxidant, prevents autoimmune disease.

In some embodiments, administering to the human subject an effective amount of the nitroxide antioxidant results in an increased expression level of a gene, for example a gene associated with the cytochrome p450 family. The gene associated with the cytochrome p450 family can be Cyp2c29, Cyp3a25, Cyp3a11, Cyp2j5, Cyp2c50, Cyp2c55, Cyp2d9, Cyp2e1, Cyp2b9, Cyp3a13, Cyp4f15, Cyp2c70, Cyp46a1, Cyp27a1, or Cyp4v3. The treatment of the human subject with the effective amount of the nitroxide antioxidant results in an increased expression level of the gene. For example, the treatment results in increased expression levels of Cyp2c29, Cyp3a25, Cyp3a11, Cyp2j5, Cyp2c50, Cyp2c55, Cyp2d9, Cyp2e1, Cyp2b9, Cyp3a13, Cyp4f15, Cyp2c70, Cyp46a1, Cyp27a1, Cyp4v3, or any combination thereof. The increased expression levels of Cyp2c29, Cyp3a25, Cyp3a11, Cyp2j5, Cyp2c50, Cyp2c55, Cyp2d9, Cyp2e1, Cyp2b9, Cyp3a13, Cyp4f15, Cyp2c70, Cyp46a1, Cyp27a1, Cyp4v3, or any combination thereof, can increase the level of apoptosis. The increased level of apoptosis results in a decrease in or disappearance of signs and symptoms of the autoimmune disease, including the curing of the autoimmune disease. In some embodiments, the increased expression levels of Cyp2c29, Cyp3a25, Cyp3a11, Cyp2j5, Cyp2c50, Cyp2c55, Cyp2d9, Cyp2e1, Cyp2b9, Cyp3a13, Cyp4f15, Cyp2c70, Cyp46a1, Cyp27a1, Cyp4v3, or any combination thereof, can decrease the level of apoptosis. The decreased level of apoptosis results in a decrease in or disappearance of signs and symptoms of the autoimmune disease, including the prevention of the autoimmune disease development.

### Methods for Treating Autoimmune Diseases

Some embodiments disclosed herein provide methods for treating an autoimmune disease in a human subject in need thereof, comprising identifying a human subject having an autoimmune disease and in need of an increased expression level of a gene associated with the cytochrome p450 family; and administering to the human subject an effective amount of a nitroxide antioxidant. In some embodiments, the methods disclosed herein may be used to treat a human subject shows no symptoms of an autoimmune disease, but is at risk of having an autoimmune disease. Exemplary risk factors for an autoimmune disease include, but are not limited to, age, family history, health conditions, medical history, habits, or a combination thereof. In some embodiments, risk factors for an autoimmune disease comprise a decreased expression level of one or more genes associated with the cytochrome p450 family.

In some embodiments, Autoimmunity is the system of immune responses of an organism against its own healthy cells and tissues. Any disease that results from such an aberrant immune response is termed an “autoimmune disease”. Prominent examples include celiac disease, diabetes mellitus type 1, sarcoidosis, systemic lupus erythematosus (SLE), Sjogren's syndrome, eosinophilic granulomatosis with polyangiitis, Hashimoto's thyroiditis, Graves' disease, idiopathic thrombocytopenic purpura, Addison's disease, rheumatoid arthritis (RA), ankylosing spondylitis, polymyositis (PM), and dermatomyositis (DM). Autoimmune diseases are very often treated with steroids

Our understanding of the role of T cells in human disease is undergoing revision as a result of the discovery of T-helper 17 (Th17) cells, a unique CD4+ T-cell subset characterized by production of interleukin-17 (IL-17). IL-17 is a highly inflammatory cytokine with robust effects on stromal cells in many tissues. Recent data in humans and mice suggest that Th17 cells play an important role in the pathogenesis of a diverse group of immune-mediated diseases, including psoriasis, rheumatoid arthritis, multiple sclerosis, inflammatory bowel disease, and asthma. Initial reports also propose a role for Th17 cells in tumorigenesis and transplant rejection. Important differences, as well as many similarities, are emerging when the biology of Th17 cells in the mouse is compared with corresponding phenomena in humans. As our understanding of human Th17 biology grows, the mechanisms underlying many diseases are becoming more apparent, resulting in a new appreciation for both previously known and more recently discovered cytokines, chemokines, and feedback mechanisms. Given the strong association between excessive Th17 activity and human disease, new therapeutic approaches targeting Th17 cells are highly promising, but the potential safety of such treatments may be limited by the role of these cells in normal host defenses against infection.

In some embodiments, the autoimmune disease is a manifestation of unregulated pathogenic activity of helper T-cells, mediated by one or more effector molecules. Helper T-cells are those differentiated from native CD4+ and classified in one or more subsets. Upon antigenic stimulation, naïve CD4+ T cells activate, expand and differentiate into different effector phenotypes. TH17 cells, which have been characterized as an additional effector T cell subset that produce interleukin (IL) glycoproteins IL-17A, IL-17F, IL-21 and IL-22, are known to be the critical driver of autoimmune tissue inflammation TH17 has been identified as having non-pathogenic and pathogenic function in the presence of effector cells or effector molecules IL-1 beta, IL-6, and IL-23.

In some embodiments, administering to the human subject an effective amount of the nitroxide antioxidant results in an increased expression level of a gene, for example a gene associated with the cytochrome p450 family. The gene associated with the cytochrome p450 family can be Cyp2c29, Cyp3a25, Cyp3a11, Cyp2j5, Cyp2c50, Cyp2c55, Cyp2d9, Cyp2e1, Cyp2b9, Cyp3a13, Cyp4f15, Cyp2c70, Cyp46a1, Cyp27a1, or Cyp4v3. The treatment of the human subject with the effective amount of the nitroxide antioxidant results in an increased expression level of the gene. For example, the treatment results in increased expression levels of Cyp2c29, Cyp3a25, Cyp3a11, Cyp2j5, Cyp2c50, Cyp2c55, Cyp2d9, Cyp2e1, Cyp2b9, Cyp3a13, Cyp4f15, Cyp2c70, Cyp46a1, Cyp27a1, Cyp4v3, or any combination thereof. The increased expression levels of Cyp2c29, Cyp3a25, Cyp3a11, Cyp2j5, Cyp2c50, Cyp2c55, Cyp2d9, Cyp2e1, Cyp2b9, Cyp3a13, Cyp4f15, Cyp2c70, Cyp46a1, Cyp27a1, Cyp4v3, or any combination thereof, can increase the level of apoptosis. The increased level of apoptosis results in a decrease in or disappearance of signs and symptoms of the autoimmune disease, including the curing of the autoimmune disease. In some embodiments, the increased expression levels of Cyp2c29, Cyp3a25, Cyp3a11, Cyp2j5, Cyp2c50, Cyp2c55, Cyp2d9, Cyp2e1, Cyp2b9, Cyp3a13, Cyp4f15, Cyp2c70, Cyp46a1, Cyp27a1, Cyp4v3, or any combination thereof, can decrease the level of apoptosis. The decreased level of apoptosis results in a decrease in or disappearance of signs and symptoms of the autoimmune disease, including the curing of the autoimmune disease.

Non-limiting examples of autoimmune diseases include rheumatoid arthritis, osteoarthritis, juvenile chronic arthritis, septic arthritis, Lyme arthritis, psoriatic arthritis, reactive arthritis, spondyloarthropathy, systemic lupus erythematosus, Crohn's disease, ulcerative colitis, inflammatory bowel disease, insulin dependent diabetes mellitus, thyroiditis, asthma, allergic diseases, psoriasis, dermatitis scleroderma, graft versus host disease, organ transplant rejection, acute or chronic immune disease associated with organ transplantation, sarcoidosis, atherosclerosis, disseminated intravascular coagulation, Kawasaki's disease, Grave's disease, nephrotic syndrome, chronic fatigue syndrome, Wegener's granulomatosis, Henoch-Schoenlein purpurea, microscopic vasculitis of the kidneys, chronic active hepatitis, uveitis, septic shock, toxic shock syndrome, sepsis syndrome, cachexia, infectious diseases, parasitic diseases, acquired immunodeficiency syndrome, acute transverse myelitis, Huntington's chorea, Parkinson's disease, Alzheimer's disease, stroke, primary biliary cirrhosis, hemolytic anemia, malignancies, heart failure, myocardial infarction, Addison's disease, sporadic, polyglandular deficiency type I and polyglandular deficiency type II, Schmidt's syndrome, adult (acute) respiratory distress syndrome, alopecia, alopecia greata, seronegative arthopathy, arthropathy, Reiter's disease, psoriatic arthropathy, ulcerative colitic arthropathy, enteropathic synovitis, chlamydia, yersinia and salmonella associated arthropathy, spondyloarthopathy, atheromatous disease/arteriosclerosis, atopic allergy, autoimmune bullous disease, pemphigus vulgaris, pemphigus foliaceus, pemphigoid, linear IgA disease, autoimmune haemolytic anaemia, Coombs positive haemolytic anaemia, acquired pernicious anaemia, juvenile pernicious anaemia, myalgic encephalitis/Royal Free Disease, chronic mucocutaneous candidiasis, giant cell arteritis, primary sclerosing hepatitis, cryptogenic autoimmune hepatitis, Acquired Immunodeficiency Disease Syndrome, Acquired Immunodeficiency Related Diseases, Hepatitis B, Hepatitis C, common varied immunodeficiency (common variable hypogammaglobulinaemia), dilated cardiomyopathy, female infertility, ovarian failure, premature ovarian failure, fibrotic lung disease, cryptogenic fibrosing alveolitis, post-inflammatory interstitial lung disease, interstitial pneumonitis, connective tissue disease associated interstitial lung disease, mixed connective tissue disease associated lung disease, systemic sclerosis associated interstitial lung disease, rheumatoid arthritis associated interstitial lung disease, systemic lupus erythematosus associated lung disease, dermatomyositis/polymyositis associated lung disease, Sjogren's disease associated lung disease, ankylosing spondylitis associated lung disease, vasculitic diffuse lung disease, haemosiderosis associated lung disease, drug-induced interstitial lung disease, fibrosis, radiation fibrosis, bronchiolitis obliterans, chronic eosinophilic pneumonia, lymphocytic infiltrative lung disease, postinfectious interstitial lung disease, gouty arthritis, autoimmune hepatitis, type-1 autoimmune hepatitis (classical autoimmune or lupoid hepatitis), type-2 autoimmune hepatitis (anti-LKM antibody hepatitis), autoimmune mediated hypoglycaemia, type B insulin resistance with acanthosis nigricans, hypoparathyroidism, acute immune disease associated with organ transplantation, chronic immune disease associated with organ transplantation, osteoarthrosis, primary sclerosing cholangitis, psoriasis type 1, psoriasis type 2, idiopathic leucopaenia, autoimmune neutropaenia, renal disease NOS, glomerulonephritides, microscopic vasulitis of the kidneys, lyme disease, discoid lupus erythematosus, male infertility idiopathic or NOS, sperm autoimmunity, multiple sclerosis (all subtypes), sympathetic ophthalmia, pulmonary hypertension secondary to connective tissue disease, Goodpasture's syndrome, pulmonary manifestation of polyarteritis nodosa, acute rheumatic fever, rheumatoid spondylitis, Still's disease, systemic sclerosis, Sjogren's syndrome, Takayasu's disease/arteritis, autoimmune thrombocytopaenia, idiopathic thrombocytopaenia, autoimmune thyroid disease, hyperthyroidism, goitrous autoimmune hypothyroidism (Hashimoto's disease), atrophic autoimmune hypothyroidism, primary myxoedema, phacogenic uveitis, primary vasculitis, vitiligo acute liver disease, chronic liver diseases, alcoholic cirrhosis, alcohol-induced liver injury, choleosatatis, idiosyncratic liver disease, Drug-Induced hepatitis, Non-alcoholic Steatohepatitis, allergy and asthma, group B streptococci (GB S) infection, mental disorders (e.g., depression and schizophrenia), Th2 Type and Th1 Type mediated diseases, acute and chronic pain (different forms of pain), and cancers such as lung, breast, stomach, bladder, colon, pancreas, ovarian, prostate and rectal cancer and hematopoietic malignancies (leukemia and lymphoma). The human antibodies, and antibody portions of the present application can be used to treat humans suffering from autoimmune diseases, in particular those associated with inflammation, including, rheumatoid spondylitis, allergy, autoimmune diabetes, autoimmune uveitis.

Non-limiting examples of autoimmune diseases include acquired immunodeficiency disease syndrome (AIDS), autoimmune lymphoproliferative syndrome, hemolytic anemia, inflammatory diseases, and thrombocytopenia, acute or chronic immune disease associated with organ transplantation, Addison's disease, allergic diseases, alopecia, alopecia areata, atheromatous disease/arteriosclerosis, atherosclerosis, arthritis (including osteoarthritis, juvenile chronic arthritis, septic arthritis, Lyme arthritis, psoriatic arthritis and reactive arthritis), autoimmune bullous disease, abetalipoprotemia, acquired immunodeficiency-related diseases, acute immune disease associated with organ transplantation, acquired acrocyanosis, acute and chronic parasitic or infectious processes, acute pancreatitis, acute renal failure, acute rheumatic fever, acute transverse myelitis, adenocarcinomas, aerial ectopic beats, adult (acute) respiratory distress syndrome, AIDS dementia complex, alcoholic cirrhosis, alcohol-induced liver injury, alcohol-induced hepatitis, allergic conjunctivitis, allergic contact dermatitis, allergic rhinitis, allergy and asthma, allograft rejection, alpha-1-antitrypsin deficiency, Alzheimer's disease, amyotrophic lateral sclerosis, anemia, angina pectoris, ankylosing spondylitis associated lung disease, anterior horn cell degeneration, antibody mediated cytotoxicity, antiphospholipid syndrome, anti-receptor hypersensitivity reactions, aortic and peripheral aneurysms, aortic dissection, arterial hypertension, arteriosclerosis, arteriovenous fistula, arthropathy, asthenia, asthma, ataxia, atopic allergy, atrial fibrillation (sustained or paroxysmal), atrial flutter, atrioventricular block, atrophic autoimmune hypothyroidism, autoimmune haemolytic anaemia, autoimmune hepatitis, type-1 autoimmune hepatitis (classical autoimmune or lupoid hepatitis), autoimmune mediated hypoglycaemia, autoimmune neutropaenia, autoimmune thrombocytopaenia, autoimmune thyroid disease, B cell lymphoma, bone graft rejection, bone marrow transplant (BMT) rejection, bronchiolitis obliterans, bundle branch block, burns, cachexia, cardiac arrhythmias, cardiac stun syndrome, cardiac tumors, cardiomyopathy, cardiopulmonary bypass inflammation response, cartilage transplant rejection, cerebellar cortical degenerations, cerebellar disorders, chaotic or multifocal atrial tachycardia, chemotherapy associated disorders, chlamydia, choleosatatis, chronic alcoholism, chronic active hepatitis, chronic fatigue syndrome, chronic immune disease associated with organ transplantation, chronic eosinophilic pneumonia, chronic inflammatory pathologies, chronic mucocutaneous candidiasis, chronic obstructive pulmonary disease (COPD), chronic salicylate intoxication, colorectal common varied immunodeficiency (common variable hypogammaglobulinaemia), conjunctivitis, connective tissue disease associated interstitial lung disease, contact dermatitis, Coombs positive haemolytic anaemia, cor pulmonale, Creutzfeldt-Jakob disease, cryptogenic autoimmune hepatitis, cryptogenic fibrosing alveolitis, culture negative sepsis, cystic fibrosis, cytokine therapy associated disorders, Crohn's disease, dementia pugilistica, demyelinating diseases, dengue hemorrhagic fever, dermatitis, dermatitis scleroderma, dermatologic conditions, dermatomyositis/polymyositis associated lung disease, diabetes, diabetic arteriosclerotic disease, diabetes mellitus, Diffuse Lewy body disease, dilated cardiomyopathy, dilated congestive cardiomyopathy, discoid lupus erythematosus, disorders of the basal ganglia, disseminated intravascular coagulation, Down's Syndrome in middle age, drug-induced interstitial lung disease, drug-induced hepatitis, drug-induced movement disorders induced by drugs which block CNS dopamine, receptors, drug sensitivity, eczema, encephalomyelitis, endocarditis, endocrinopathy, enteropathic synovitis, epiglottitis, Epstein-Barr virus infection, erythromelalgia, extrapyramidal and cerebellar disorders, familial hematophagocytic lymphohistiocytosis, fetal thymus implant rejection, Friedreich's ataxia, functional peripheral arterial disorders, female infertility, fibrosis, fibrotic lung disease, fungal sepsis, gas gangrene, gastric ulcer, giant cell arteritis, glomerular nephritis, glomerulonephritides, Goodpasture's syndrome, goitrous autoimmune hypothyroidism (Hashimoto's disease), gouty arthritis, graft rejection of any organ or tissue, graft versus host disease, gram negative sepsis, gram positive sepsis, granulomas due to intracellular organisms, group B streptococci (GBS) infection, Grave's disease, haemosiderosis associated lung disease, hairy cell leukemia, hairy cell leukemia, Hallerrorden-Spatz disease, Hashimoto's thyroiditis, hay fever, heart transplant rejection, hemachromatosis, hematopoietic malignancies (leukemia and lymphoma), hemolytic anemia, hemolytic uremic syndrome/thrombolytic thrombocytopenic purpura, hemorrhage, Henoch-Schoenlein purpurea, Hepatitis A, Hepatitis B, Hepatitis C, HIV infection/HIV neuropathy, Hodgkin's disease, hypoparathyroidism, Huntington's chorea, hyperkinetic movement disorders, hypersensitivity reactions, hypersensitivity pneumonitis, hyperthyroidism, hypokinetic movement disorders, hypothalamic-pituitary-adrenal axis evaluation, idiopathic Addison's disease, idiopathic leucopaenia, idiopathic pulmonary fibrosis, idiopathic thrombocytopaenia, idiosyncratic liver disease, infantile spinal muscular atrophy, infectious diseases, inflammation of the aorta, inflammatory bowel disease, insulin dependent diabetes mellitus, interstitial pneumonitis, iridocyclitis/uveitis/optic neuritis, ischemia-reperfusion injury, ischemic stroke, juvenile pernicious anaemia, juvenile rheumatoid arthritis, juvenile spinal muscular atrophy, Kaposi's sarcoma, Kawasaki's disease, kidney transplant rejection, legionella, leishmaniasis, leprosy, lesions of the corticospinal system, linear IgA disease, lipidema, liver transplant rejection, Lyme disease, lymphederma, lymphocytic infiltrative lung disease, malaria, male infertility idiopathic or NOS, malignant histiocytosis, malignant melanoma, meningitis, meningococcemia, microscopic vasculitis of the kidneys, migraine headache, mitochondrial multisystem disorder, mixed connective tissue disease, mixed connective tissue disease associated lung disease, monoclonal gammopathy, multiple myeloma, multiple systems degenerations (Mencel Dejerine-Thomas Shi-Drager and Machado-Joseph), myalgic encephalitis/Royal Free Disease, myasthenia gravis, microscopic vasculitis of the kidneys, mycobacterium avium intracellulare, mycobacterium tuberculosis, myelodyplastic syndrome, myocardial infarction, myocardial ischemic disorders, nasopharyngeal carcinoma, neonatal chronic lung disease, nephritis, nephrosis, nephrotic syndrome, neurodegenerative diseases, neurogenic I muscular atrophies, neutropenic fever, Non-alcoholic Steatohepatitis, occlusion of the abdominal aorta and its branches, occlusive arterial disorders, organ transplant rejection, orchitis/epidydimitis, orchitis/vasectomy reversal procedures, organomegaly, osteoarthrosis, osteoporosis, ovarian failure, pancreas transplant rejection, parasitic diseases, parathyroid transplant rejection, Parkinson's disease, pelvic inflammatory disease, pemphigus vulgaris, pemphigus foliaceus, pemphigoid, perennial rhinitis, pericardial disease, peripheral atherlosclerotic disease, peripheral vascular disorders, peritonitis, pernicious anemia, phacogenic uveitis, pneumocystis carinii pneumonia, pneumonia, POEMS syndrome (polyneuropathy, organomegaly, endocrinopathy, monoclonal gammopathy, and skin changes syndrome), post perfusion syndrome, post pump syndrome, post-MI cardiotomy syndrome, postinfectious interstitial lung disease, premature ovarian failure, primary biliary cirrhosis, primary sclerosing hepatitis, primary myxoedema, primary pulmonary hypertension, primary sclerosing cholangitis, primary vasculitis, Progressive supranucleo Palsy, psoriasis, psoriasis type 1, psoriasis type 2, psoriatic arthropathy, pulmonary hypertension secondary to connective tissue disease, pulmonary manifestation of polyarteritis nodosa, post-inflammatory interstitial lung disease, radiation fibrosis, radiation therapy, Raynaud's phenomenon and disease, Raynoud's disease, Refsum's disease, regular narrow QRS tachycardia, Reiter's disease, renal disease NOS, renovascular hypertension, reperfusion injury, restrictive cardiomyopathy, rheumatoid arthritis associated interstitial lung disease, rheumatoid spondylitis, sarcoidosis, Schmidt's syndrome, scleroderma, senile chorea, Senile Dementia of Lewy body type, sepsis syndrome, septic shock, seronegative arthropathies, shock, sickle cell anemia, Sjogren's disease associated lung disease, Sjorgren's syndrome, skin allograft rejection, skin changes syndrome, small bowel transplant rejection, sperm autoimmunity, multiple sclerosis (all subtypes), spinal ataxia, spinocerebellar degenerations, spondyloarthropathy, spondyloarthopathy, sporadic, polyglandular deficiency type I sporadic, polyglandular deficiency type II, Still's disease, streptococcal myositis, stroke, structural lesions of the cerebellum, Subacute sclerosing panencephalitis, sympathetic ophthalmia, Syncope, syphilis of the cardiovascular system, systemic anaphylaxis, systemic inflammatory response syndrome, systemic onset juvenile rheumatoid arthritis, systemic lupus erythematosus, systemic lupus erythematosus-associated lung disease, systemic sclerosis, systemic sclerosis-associated interstitial lung disease, T-cell or FAB ALL, Takayasu's disease/arteritis, Telangiectasia, Th2 Type and Th1 Type mediated diseases, thromboangitis obliterans, thrombocytopenia, thyroiditis, toxicity, toxic shock syndrome, transplants, trauma/hemorrhage, type-2 autoimmune hepatitis (anti-LKM antibody hepatitis), type B insulin resistance with acanthosis nigricans, type III hypersensitivity reactions, type IV hypersensitivity, ulcerative colitic arthropathy, ulcerative colitis, unstable angina, uremia, urosepsis, urticaria, uveitis, valvular heart diseases, varicose veins, vasculitis, vasculitic diffuse lung disease, venous diseases, venous thrombosis, ventricular fibrillation, vitiligo acute liver disease, viral and fungal infections, vital encephalitis/aseptic meningitis, vital-associated hemaphagocytic syndrome, Wegener's granulomatosis, Wernicke-Korsakoff syndrome, Wilson's disease, xenograft rejection of any organ or tissue, yersinia and salmonella-associated arthropathy and the like.

### Nitroxide Antioxidant

Nitroxide antioxidants describes a group of stable organic molecules, containing the nitroxyl group >N—O· with an unpaired electron. They have a low molecular weight, are non-toxic, do not elicit immunogenic effects on cells and easily diffuse through cell membranes. Their biological activity as antioxidants is related to the regulation of redox state in the cells. Nitroxides can undergo cyclic oxidation or reduction reactions. Their antioxidant activity is related to several mechanisms such as the direct scavenging of free radicals, transition metal ion oxidation. In addition, nitroxides exhibit superoxide dismutase (SOD)-like activity, modulate its catalase-like activity and ferroxidase-like activity, and are the inhibitors of free radical reactions such as lipid peroxidation. Nitroxides have dynamic beneficial impact on all cellular processes from inhibition of oxidative stress and reducing inflammation, while under certain conditions they may also lead to its intensification, for example, in tumor cells. The different beneficial impact on cellular processes provides each cell with necessary support to prevent or reverse diseases and conditions through optimizing cellular activity and associated biological processes in a healthy state and promoting cell death in diseases such as cancer.

Cyclic nitroxides, also known as aminoxyls or nitroxyls, are stable free radicals stabilized by methyl groups at the a position in five-membered pyrrolidine, pyrroline or oxazolidine and six-membered piperidine ring structures. The methyl groups confer stability to the nitroxide radicals by preventing radical-radical dismutation and also limit access to reactive substances, which can quench the radical species. The substituent groups on the ring (denoted by R—) produce a diverse range of compounds that can be directed to specific hydrophilic or hydrophobic regions in the cellular microenvironment. The redox transformations between the oxidation states of nitroxide, hydroxylamine and the oxoammonium cation acts as an efficient redox couple, which can support catalytic processes via reversible electron redox reactions. (Soule, Benjamin P et al. “The chemistry and biology of nitroxide compounds.” Free radical biology & medicine vol. 42,11 (2007): 1632-50. doi: 10.1016/j.freeradbiomed.2007.02.030).

The mechanism of action exerted by nitroxide antioxidants is very unique. In particular, nitroxide antioxidant function is characterized by a catalytic mechanism of action associated with a single-electron redox cycle. Their reduction results in the generation of hydroxylamine and oxidation in oxoammonium ion; meanwhile both reactions are reversible and repetitive such that the ratio of free radicals suppressed by nitroxide antioxidants is significantly higher than natural antioxidant processes within a cell. Hydroxylamine also exhibits antioxidant properties because it is easily oxidized to nitroxide. As mentioned above, the nitroxides devoid of electrical charge easily diffuse through the cell membranes, thus they can also inactivate the reactive oxygen species formed in the cells and modulate the concentration of intracellular nitric oxide. Their molecular structure and composition make nitroxide antioxidants additionally efficacious in tissues that prevent transport of different molecules, such as neuronal tissue across the blood brain barrier.

Non-limiting examples of the nitroxide antioxidant include 2-ethyl-2,5,5-trimethyl-3-oxazolidine-1-oxyl (OXANO), 2,2,6,6-tetramethylpiperidine-1-oxyl (TEMPO), 4-hydroxy-2,2,6,6-tetramethylpiperidine-1-oxyl (TEMPOL), 4-amino-2,2,6,6-tetramethyl-1-piperidinyloxy (Tempamine), 3-Amin omethyl-PROXYL, 3-Cyano-PROXYL, 3-Carbamoyl-PROXYL, 3-Carboxy-PROXYL, and 4-Oxo-TEMPO. TEMPO can also be substituted, typically in the 4 position, for example, 4-amino, 4-(2-bromoacetamido), 4-(ethoxyfluorophosphonyloxy), 4-hydroxy, 4-(2-iodoacetamido), 4-isothiocyanato, 4-maleimido, 4-(4-nitrobenzoyloxyl), 4-phosphonooxy, 2,2,6,6-tetramethyl-4-oxo-1-piperidinyloxy (TEMPONE), 1-Hydroxy-2,2,6,6-tetramethyl-4-oxo-piperidine. HCl (TEMPONE-H), 1,2-dipalmitoyl-sn-glycero-3-phospho(tempo)choline (TEMPO PC), (4-[N,N-dimethyl-N-(2-hydroxyethyl)]ammonium-2,2,6,6-tetramethylpiperidine-1-oxyl (TEMPO Choline), and the like.

The use of other nitroxide compounds is also contemplated. Nitroxide stable radicals demonstrate effective antioxidative activity in various biological systems ranging from molecular, cellular, and laboratory animal level. Nitroxides have been reported to catalyze O2. dismutation through two different catalytic pathways including reductive and oxidative reaction mechanisms. Conversely, kinetics analysis of rapid mixing stopped flow experiments de-signed to measure the effect of nitroxides on superoxide decay did not reveal any SOD activity, leading to the conclusion that nitroxides act as free radical scavengers.

Studies have shown that unlike other antioxidants, nitroxides are characterized by a catalytic mechanism of action associated with a single-electron redox cycle. Their reduction results in the generation of hydroxylamine and oxidation in oxoammonium ion; meanwhile both reactions are reversible. Hydroxylamine also exhibits antioxidant properties because it is easily oxidized to nitroxide. Nitroxide antioxidants undergo redox cycles. They are easily reduced to hydroxylamines and oxidized to oxoammonium salts.

According to certain embodiments the nitroxide compound can be selected from the following formulas:

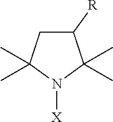

wherein X is selected from O— and OH, and R is selected from COOH, CONH, CN, and CH2NH2;

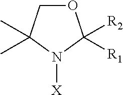

wherein X is selected from O— and OH, and R1 is selected from CH3 and spirocyclohexyl, and R2 is selected from C2H5 and spirocyclohexyl;

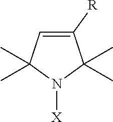

wherein X is selected from O— and OH and R is selected from CONH; and

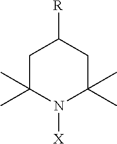

wherein X is selected from O— and OH and R is selected from H, OH, and NH2.

Suitable nitroxide compounds can also be found in Proctor, U.S. Pat. No. 5,352,442, and Mitchell et al., U.S. Pat. No. 5,462,946, both of which are hereby incorporated by reference in their entireties.

In some embodiments, the nitroxide antioxidant has a general formula:

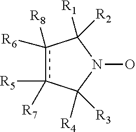

wherein the dashed line denotes a saturated bond or an unsaturated bond, wherein when the dashed line denotes an unsaturated bond, R7 and R8 are absent; R1-R4 are each independently a C1-4-alkyl, or alternatively, R1 and R2, and/or R3 and R4, together form a 3-7-membered alicyclic ring; and R5-R8 are each independently selected from the group consisting of hydrogen, alkyl, alkenyl, alkynyl, cycloalkyl, aryl, heteroaryl, heteroalicyclic, halo, hydroxy, alkoxy, aryloxy, thiohydroxy, thioalkoxy, thioaryloxy, sulfinyl, sulfonyl, sulfonate, sulfate, cyano, nitro, azide, phosphonyl, phosphinyl, carbonyl, thiocarbonyl, urea, thiourea, O-carbamyl, N-carbamyl, O-thiocarbamyl, N-thiocarbamyl, C-amido, N-amido, C-carboxy, O-carboxy, sulfonamido, hydrazine, and amino,

In some embodiments, the nitroxide antioxidant includes or is associated with (e.g., binds to or is conjugated with) a bioeffector molecule. For example, the bioeffector molecule is a targeting subunit bound to the nitroxide antioxidant, such as a mitochondrial targeting subunit. A targeting subunit can direct activity of the nitroxide antioxidant to a predetermined location within or on the cell. Non-limiting examples of mitochondrial targeting bioeffector molecules includes triphenylphosphine (TPP), gramicidin, and any functional group effectively charged to be attracted to the polarized mitochondria.

In some embodiments, the nitroxide antioxidant is structurally cyclic having a ring structure including a nitroxide molecule incorporated therein. In some embodiments, the nitroxide antioxidant is characterized as the nitroxide molecule functioning as the catalytic center.

### Dosage

In some embodiments, the nitroxide antioxidant, non-toxic salts thereof, acid addition salts thereof or hydrates thereof may be administered systemically or locally, usually by oral or parenteral administration. The doses to be administered can be determined depending upon, for example, age, body weight, symptom, the desired therapeutic effect, the route of administration, and the duration of the treatment. In the human adult, the dose per person at a time can be generally from about 0.01 to about 4000 mg, by oral administration, up to several times per day. Specific examples of particular amounts contemplated via oral administration include about 0.02, 0.03, 0.04, 0.05, 0.10, 0.15, 0.20, 0.25, 0.30, 0.35, 0.40, 0.45, 0.50, 0.55, 0.60, 0.65, 0.70, 0.75, 0.80, 0.85, 0.90, 0.95, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 405, 410, 415, 420, 425, 430, 435, 440, 445, 450, 455, 460, 465, 470, 475, 480, 485, 490, 495, 500, 505, 510, 515, 520, 525, 530, 535, 540, 545, 550, 555, 560, 565, 570, 575, 580, 585, 590, 595, 600, 605, 610, 615, 620, 625, 630, 635, 640, 645, 650, 655, 660, 665, 670, 675, 680, 685, 690, 695, 700, 705, 710, 715, 720, 725, 730, 735, 740, 745, 750, 755, 760, 765, 770, 775, 780, 785, 790, 795, 800, 805, 810, 820, 825, 830, 835, 840, 845, 850, 855, 860, 865, 870, 875, 880, 885, 890, 895, 900, 905, 910, 915, 920, 925, 930, 935, 940, 945, 950, 955, 960, 965, 970, 975, 980, 985, 990, 995, 1000 or more mg. The dose per person at a time can be generally from about 0.01 to about 300 mg/kg via parenteral administration (preferably intravenous administration), from one to four or more times per day. Specific examples of particular amounts contemplated include about 0.02, 0.03, 0.04, 0.05, 0.10, 0.15, 0.20, 0.25, 0.30, 0.35, 0.40, 0.45, 0.50, 0.55, 0.60, 0.65, 0.70, 0.75, 0.80, 0.85, 0.90, 0.95, 1, 2, 3, 4, 5, 6, 7, 8, 9, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300 or more mg/kg. Continuous intravenous administration can also contemplated for from 1 to 24 hours per day to achieve a target concentration from about 0.01 mg/L to about 100 mg/L. Non-limiting examples of particular amounts contemplated via this route include about 0.02, 0.03, 0.04, 0.05, 0.10, 0.15, 0.20, 0.25, 0.30, 0.35, 0.40, 0.45, 0.50, 0.55, 0.60, 0.65, 0.70, 0.75, 0.80, 0.85, 0.90, 0.95, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more mg/L. The dose to be used does can depend upon various conditions, and there may be cases wherein doses lower than or greater than the ranges specified above are used.

### Compositions

The nitroxide antioxidant can be administered in the form of, for example, solid compositions, liquid compositions or other compositions for oral administration, injections, liniments or suppositories for parenteral administration.

Solid compositions for oral administration include compressed tablets, pills, capsules, dispersible powders and granules. Capsules include hard capsules and soft capsules. In such solid compositions, Tempol may be admixed with an excipient (e.g. lactose, mannitol, glucose, microcrystalline cellulose, starch), combining agents (hydroxypropyl cellulose, polyvinyl pyrrolidone or magnesium metasilicate aluminate), disintegrating agents (e.g. cellulose calcium glycolate), lubricating agents (e.g. magnesium stearate), stabilizing agents, agents to assist dissolution (e.g. glutamic acid or aspartic acid), or the like. The agents may, if desired, be coated with coating agents (e.g. sugar, gelatin, hydroxypropyl cellulose or hydroxypropylmethyl cellulose phthalate), or be coated with two or more films. Further, coating may include containment within capsules of absorbable materials such as gelatin.

Liquid compositions for oral administration include pharmaceutically acceptable solutions, suspensions, emulsions, syrups and elixirs. In such compositions, the nitroxide antioxidant is dissolved, suspended or emulsified in a commonly used diluent (e.g. purified water, ethanol or mixture thereof). Furthermore, such liquid compositions may also comprise wetting agents or suspending agents, emulsifying agents, sweetening agents, flavoring agents, perfuming agents, preserving agents, buffer agents, or the like.

Injections for parenteral administration include solutions, suspensions, emulsions and solids which are dissolved or suspended. For injections, the nitroxide antioxidant can be dissolved, suspended and emulsified in a solvent. The solvents include, for example, distilled water for injection, physiological salt solution, vegetable oil, propylene glycol, polyethylene glycol, alcohol such as ethanol, or a mixture thereof. Moreover the injections can also include stabilizing agents, agents to assist dissolution (e.g. glutamic acid, aspartic acid or POLYSORBATE80™), suspending agents, emulsifying agents, soothing agents, buffer agents, preserving agents, etc. They can be sterilized in the final process or manufactured and prepared by sterile procedure. They can also be manufactured in the form of sterile solid compositions, such as a freeze-dried composition, and they may be sterilized or dissolved immediately before use in sterile distilled water for injection or some other solvent.

Other compositions for parenteral administration include liquids for external use, and ointment, endermic liniments, inhale, spray, suppositories for rectal administration and pessaries for vaginal administration which comprise the nixtroxide antioxidant and are administered by methods known in the art.

Spray compositions can comprise additional substances other than diluents: e.g. stabilizing agents (e.g. sodium sulfite hydride), isotonic buffers (e.g. sodium chloride, sodium citrate or citric acid). A small aerosol particle size useful for effective distribution of the medicament can be obtained by employing self-propelling compositions containing the drugs in micronized form dispersed in a propellant composition. Effective dispersion of the finely divided drug particles can be accomplished with the use of very small quantities of a suspending agent, present as a coating on the micronized drug particles. Evaporation of the propellant from the aerosol particles after spraying from the aerosol container leaves finely divided drug particles coated with a fine film of the suspending agent. In the micronized form, the average particle size can be less than about 5 microns. The propellant composition may employ, as the suspending agent, a fatty alcohol such as oleyl alcohol. The minimum quantity of suspending agent can be approximately 0.1 to 0.2 percent by weight of the total composition. The amount of suspending agent can be less than about 4 percent by weight of the total composition to maintain an upper particle size limit of less than 10 microns or 5 microns. Propellants that may be employed include hydrofluoroalkane propellants and chlorofluorocarbon propellants. Dry powder inhalation may also be employed.

### EXAMPLES

The following examples are offered to illustrate but not to limit the invention.

In order to facilitate understanding, the specific embodiments are provided to help interpret the technical proposal, that is, these embodiments are only for illustrative purposes, but not in any way to limit the scope of the invention. Unless otherwise specified, embodiments do not indicate the specific conditions, are in accordance with the conventional conditions or the manufacturer's recommended conditions.

### Example 1. Effects of Tempol on Expression of Genes Associated With the Cytochrome p450 Family

To assess the effects of Tempol on gene expression, Tempol was administered to experimental mice at a dose of 5 mg/g of food from 14 months to 31 months after birth. Mice receiving the same food without the addition of Tempol were used as a negative control. At the age of 31 months, the experimental animals were sacrificed and the hearts were surgically removed. The expression of a broad spectrum of genes in the cardiac tissue was assessed using chip-based microarray technology. Such chips are well known in the art and are widely used to assess gene expression. The experimental results showed that fifteen genes associated with the cytochrome p450 family, Cyp2c29, Cyp3a25, Cyp3a11, Cyp2j5, Cyp2c50, Cyp2c55, Cyp2d9, Cyp2e1, Cyp2b9, Cyp3a13, Cyp4f15, Cyp2c70, Cyp46a1, Cyp27a1, Cyp4v3, and homologues thereof, exhibited statistically significant increase in expression. This result is shown in Table 1.

TABLE 1
Genes Associated With The cytochrome p450 family
Exhibiting Increased Expression In White Adipose
Tissue After Tempol Administration

| | | | Tempol- | | |
|---|---|---|---|---|---|
| | | Control | treated | Fold | |
| Symbol | Gene title | mice | mice | change | P-value |
| Cyp2c29 | Cytochrome P450, | 90 | 1210 | 13.45 | 0.01 |
| | family 2, | | | | |
| | subfamily c, | | | | |
| | polypeptide 29 | | | | |
| Cyp3a25 | Cytochrome P450, | 65 | 564 | 8.61 | 0.02 |
| | family 3, | | | | |
| | subfamily a, | | | | |
| | polypeptide 25 | | | | |
| Cyp3a11 | Cytochrome P450, | 177 | 1375 | 7.77 | 0.02 |
| | family 3, | | | | |
| | subfamily a, | | | | |
| | polypeptide 11 | | | | |
| Cyp2j5 | Cytochrome P450, | 26 | 138 | 5.37 | 0.04 |
| | family 2, | | | | |
| | subfamily j, | | | | |
| | polypeptide 5 | | | | |
| Cyp2c50 | Cytochrome P450, | 41 | 147 | 3.58 | 0.02 |
| | family 2, | | | | |
| | subfamily c, | | | | |
| | polypeptide 50 | | | | |
| Cyp2c55 | Cytochrome P450, | 15 | 44 | 2.97 | 0.04 |
| | family 2, | | | | |
| | subfamily c, | | | | |
| | polypeptide 55 | | | | |
| Cyp2d9 | Cytochrome P450, | 72 | 200 | 2.78 | 0.04 |
| | family 2, | | | | |
| | subfamily d, | | | | |
| | polypeptide 9 | | | | |
| Cyp2e1 | Cytochrome P450, | 2329 | 5079 | 2.18 | 0.02 |
| | family 2, | | | | |
| | subfamily e, | | | | |
| | polypeptide 1 | | | | |
| Cyp2b9 | Cytochrome P450, | 54 | 103 | 1.91 | 0.03 |
| | family 2, | | | | |
| | subfamily b, | | | | |
| | polypeptide 9 | | | | |
| Cyp3a13 | Cytochrome P450, | 50 | 92 | 1.85 | 0.04 |
| | family 3, | | | | |
| | subfamily a, | | | | |
| | polypeptide 13 | | | | |
| Cyp4f15 | Cytochrome P450, | 72 | 122 | 1.70 | 0.03 |
| | family 4, | | | | |
| | subfamily f, | | | | |
| | polypeptide 15 | | | | |
| Cyp2c70 | Cytochrome P450, | 180 | 292 | 1.62 | 0.03 |
| | family 2, | | | | |
| | subfamily c, | | | | |
| | polypeptide 70 | | | | |
| Cyp46a1 | Cytochrome P450, | 115 | 166 | 1.45 | 0.04 |
| | family 46, | | | | |
| | subfamily a, | | | | |
| | polypeptide 1 | | | | |
| Cyp27a1 | Cytochrome P450, | 864 | 1135 | 1.31 | 0.02 |
| | family 27, | | | | |
| | subfamily a, | | | | |
| | polypeptide 1 | | | | |
| Cyp4v3 | Cytochrome P450, | 739 | 901 | 1.22 | 0.04 |
| | family 4, | | | | |
| | subfamily v, | | | | |
| | polypeptide 3 | | | | |

### Example 2. Treating Age-Related Decrease in Gene Expression

A 70-kilogram human subject over the age of 65 is identified for decreased expression level of Cyp2c29, Cyp3a25, Cyp3a11, Cyp2j5, Cyp2c50, Cyp2c55, Cyp2d9, Cyp2e1, Cyp2b9, Cyp3a13, Cyp4f15, Cyp2c70, Cyp46a1, Cyp27a1, or Cyp4v3. The human subject is administered a dose of 2000 mg of Tempol per day for 180 days. This may be administered in a single dose, or may be administered as a number of smaller doses over a 24-hour period: for example, three 500-mg doses at eight-hour intervals. Following treatment, the serum level of Cyp2c29, Cyp3a25, Cyp3a11, Cyp2j5, Cyp2c50, Cyp2c55, Cyp2d9, Cyp2e1, Cyp2b9, Cyp3a13, Cyp4f15, Cyp2c70, Cyp46a1, Cyp27a1, or Cyp4v3, is increased.

### Example 3. Treating a Human Subject with Decreased Gene Expression

A 70-kilogram human subject is identified for decreased expression level of Cyp2c29, Cyp3a25, Cyp3a11, Cyp2j5, Cyp2c50, Cyp2c55, Cyp2d9, Cyp2e1, Cyp2b9, Cyp3a13, Cyp4f15, Cyp2c70, Cyp46a1, Cyp27a1, or Cyp4v3. The human subject is administered a dose of 2000 mg of Tempol per day for 180 days. This may be administered in a single dose, or may be administered as a number of smaller doses over a 24-hour period: for example, three 500-mg doses at eight-hour intervals. Following treatment, the serum level of Cyp2c29, Cyp3a25, Cyp3a11, Cyp2j5, Cyp2c50, Cyp2c55, Cyp2d9, Cyp2e1, Cyp2b9, Cyp3a13, Cyp4f15, Cyp2c70, Cyp46a1, Cyp27a1, or Cyp4v3, is increased.

### Example 4. Treating a Human Subject With Age-Related Disease

A 70-kilogram human subject over the age of 65 and having a cardiovascular disease is identified for decreased expression level of Cyp2c29, Cyp3a25, Cyp3a11, Cyp2j5, Cyp2c50, Cyp2c55, Cyp2d9, Cyp2e1, Cyp2b9, Cyp3a13, Cyp4f15, Cyp2c70, Cyp46a1, Cyp27a1, or Cyp4v3. The human subject is administered a dose of 2000 mg of Tempol per day for 180 days. This may be administered in a single dose, or may be administered as a number of smaller doses over a 24-hour period: for example, three 500-mg doses at eight-hour intervals. Following treatment, the serum level of Cyp2c29, Cyp3a25, Cyp3a1 1, Cyp2j5, Cyp2c50, Cyp2c55, Cyp2d9, Cyp2e1, Cyp2b9, Cyp3a13, Cyp4f15, Cyp2c70, Cyp46a1, Cyp27a1, or Cyp4v3, is increased.

### Example 5. Treating a Human Subject at Risk of Developing Cancer

A 70-kilogram human subject at risk of developing colorectal cancer is identified for decreased expression level of Cyp2c29, Cyp3a25, Cyp3a11, Cyp2j5, Cyp2c50, Cyp2c55, Cyp2d9, Cyp2e1, Cyp2b9, Cyp3a13, Cyp4f15, Cyp2c70, Cyp46a1, Cyp27a1, or Cyp4v3. The human subject is administered a dose of 2000 mg of Tempol per day for 180 days. This may be administered in a single dose, or may be administered as a number of smaller doses over a 24-hour period: for example, three 500-mg doses at eight-hour intervals. Following treatment, the serum level of Cyp2c29, Cyp3a25, Cyp3a11, Cyp2j5, Cyp2c50, Cyp2c55, Cyp2d9, Cyp2e1, Cyp2b9, Cyp3a13, Cyp4f15, Cyp2c70, Cyp46a1, Cyp27a1, or Cyp4v3, is increased.

### Example 6. Treating a Human Subject at Risk of Developing an Autoimmune Disease

A 70-kilogram human subject at risk of developing rheumatoid arthritis is identified for decreased expression level of Cyp2c29, Cyp3a25, Cyp3a11, Cyp2j5, Cyp2c50, Cyp2c55, Cyp2d9, Cyp2e1, Cyp2b9, Cyp3a13, Cyp4f15, Cyp2c70, Cyp46a1, Cyp27a1, or Cyp4v3. The human subject is administered a dose of 2000 mg of Tempol per day for 180 days. This may be administered in a single dose, or may be administered as a number of smaller doses over a 24-hour period: for example, three 500-mg doses at eight-hour intervals. Following treatment, the serum level of Cyp2c29, Cyp3a25, Cyp3a11, Cyp2j5, Cyp2c50, Cyp2c55, Cyp2d9, Cyp2e1, Cyp2b9, Cyp3a13, Cyp4f15, Cyp2c70, Cyp46a1, Cyp27a1, or Cyp4v3, is increased.

### Example 7. Treating a Human Subject at Risk of Developing a Condition Due to Aging

A 70-kilogram human subject of 45 years old at risk of developing a condition due to aging is identified. The human subject is administered a dose of 2000 mg of Tempol per day for 180 days. This may be administered in a single dose, or may be administered as a number of smaller doses over a 24-hour period: for example, three 500-mg doses at eight-hour intervals. Following treatment, the serum level of Cyp2c29, Cyp3a25, Cyp3a11, Cyp2j5, Cyp2c50, Cyp2c55, Cyp2d9, Cyp2e1, Cyp2b9, Cyp3a13, Cyp4f15, Cyp2c70, Cyp46a1, Cyp27a1, or Cyp4v3, is increased.

In at least some of the previously described embodiments, one or more elements used in an embodiment can interchangeably be used in another embodiment unless such a replacement is not technically feasible. It will be appreciated by those skilled in the art that various other omissions, additions and modifications may be made to the methods and structures described above without departing from the scope of the claimed subject matter. All such modifications and changes are intended to fall within the scope of the subject matter, as defined by the appended claims.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

1. A method of increasing expression level of a gene encoding one or more cytochrome p450 proteins in a subject having a condition selected from the group consisting of non-alcoholic fatty liver disease, hepatocellular carcinoma, hepatic disease, a neurodegenerative disease, cirrhosis, hepatocellular carcinoma and a family history of Alzheimer's Disease, the method comprising:
identifying a subject having a decreased expression level of the gene encoding one or more cytochrome p450 proteins associated with a condition selected from the group consisting of non-alcoholic fatty liver disease, hepatocellular carcinoma, hepatic disease, a neurodegenerative disease, cirrhosis, hepatocellular carcinoma and a family history of Alzheimer's Disease; and
administering an effective amount of a nitroxide antioxidant to the subject,
wherein the nitroxide antioxidant has the general formula 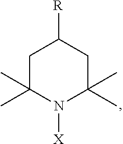
wherein x is wherein X is selected from O— and OH and R is selected from H, OH, and NH₂,
wherein the administration of the nitroxide antioxidant increases expression level of the gene encoding one or more cytochrome p450 proteins, and
wherein the increased expression level of the gene treats the condition.

2. The method of claim 1, wherein the subject has hepatocellular carcinoma.

3. (canceled)

4. (canceled)

5. The method of claim 1, wherein the subject has a hepatic disease.

6. The method of claim 1, wherein increasing the expression level prevents a neurodegenerative disease.

7. The method of claim 1, wherein the subject has cirrhosis.

8. The method of claim 1, wherein the individual has a family history of Alzheimer's Disease.

9. The method of claim 1, wherein the nitroxide antioxidant is administered before one or more chemotherapeutic agents.

10. The method of claim 1, wherein the subject has a neurodegenerative disease caused by excess brain cholesterol.

11. The method of claim 1, wherein the subject has non-alcoholic fatty liver disease.

12. The method of claim 1, wherein the nitroxide antioxidant is 4-hydroxy-2,2,6,6-tetramethylpiperidine-1-oxyl (TEMPOL).

13. A method of increasing expression level of a gene encoding one or more cytochrome p450 proteins, the method comprising:
administering an effective amount of a nitroxide antioxidant to a subject having or at risk of developing a disease associated with decreased cytochrome p450 expression,
wherein the disease is selected from the group consisting of non-alcoholic fatty liver disease, hepatocellular carcinoma, hepatic disease, a neurodegenerative disease, cirrhosis, hepatocellular carcinoma and a family history of Alzheimer's Disease,
wherein the gene encoding one or more cytochrome p450 proteins is selected from the group consisting of Cyp2c29, Cyp3a25, Cyp3a11, Cyp2j5, Cyp2c50, Cyp2c55, Cyp2d9, Cyp2e1, Cyp2b9, Cyp3a13, Cyp4f15, Cyp2c70, Cyp46a1, Cyp27a1, and Cyp4v3, and
wherein the nitroxide antioxidant increases an expression level of the gene encoding one or more cytochrome p450 proteins.

14. The method of claim 13, wherein the nitroxide antioxidant is 4-hydroxy-2,2,6,6-tetramethylpiperidine-1-oxyl (TEMPOL).

15. The method of claim 13, wherein the nitroxide antioxidant passes through the blood brain barrier.

16. The method of claim 13, wherein the disease is caused by increased cholesterol.

17. The method of claim 13, wherein the subject has hepatitis.

18. The method of claim 13, wherein the subject is in need of increased xenobiotic metabolism.

19. (canceled)

20. (Canceled)

21. The method of claim 13, wherein the nitroxide antioxidant is selected from the group consisting of 2-ethyl-2,5,5-trimethyl-3-oxazolidine-1-oxyl (OXANO), 2,2,6,6-tetramethylpiperidine-1-oxyl (TEMPO), 4-hydroxy-2,2,6,6-tetramethylpiperidine-1-oxyl (TEMPOL), 4-amino-2,2,6,6-tetramethyl-1-piperidinyloxy (Tempamine), 3-Amin omethyl-PROXYL, 3-Cyano-PROXYL, 3-Carbamoyl-PROXYL, 3-Carboxy-PROXYL, 4-Oxo-TEMPO, 2,2,6,6-tetramethyl-4-oxo-1-piperidinyloxy (TEMPONE), 1-Hydroxy-2,2,6,6-tetramethyl-4-oxo-piperidine HCl (TEMPONE-H), 1,2-dipalmitoyl-sn-glycero-3-phospho(tempo)choline (TEMPO PC), and (4-[N,N-dimethyl-N-(2-hydroxyethyl)]ammonium-2,2,6,6-tetramethylpiperidine-1-oxyl (TEMPO Choline).

22. The method of claim 13, wherein the disease is non-alcoholic fatty liver disease

23. The method of claim 1, wherein the gene encoding one or more cytochrome p450 proteins is selected from the group consisting of Cyp2c29, Cyp3a25, Cyp3a11, Cyp2j5, Cyp2c50, Cyp2c55, Cyp2d9, Cyp2e1, Cyp2b9, Cyp3a13, Cyp4f15, Cyp2c70, Cyp46a1, Cyp27a1, and Cyp4v3.

24. (Withdrawn— currently amended) The method of claim 1, wherein the nitroxide antioxidant is selected from the group consisting of 2-ethyl-2,5,5-trimethyl-3-oxazolidine-1-oxyl (OXANO), 2,2,6,6-tetramethylpiperidine-1-oxyl (TEMPO), 4-amino-2,2,6,6-tetramethyl-1-piperidinyloxy (Tempamine), 3-Amin omethyl-PROXYL, 3-Cyano-PROXYL, 3-Carbamoyl-PROXYL, 3-Carboxy-PROXYL, 4-Oxo-TEMPO. 2,2,6, 6-tetramethyl-4-oxo-1-piperidinyloxy (TEMPONE), 1-Hydroxy-2,2,6,6-tetramethyl-4-oxo-piperidine HCl (TEMPONE-H), 1,2-dipalmitoyl-sn-glycero-3-phospho(tempo)choline (TEMPO PC), and (4-[N,N- dimethyl-N-(2-hydroxyethyl)]ammonium-2,2,6,6-tetramethylpiperidine-1-oxyl (TEMPO Choline).
